(19) United States
(12) Patent Application Publication
(10) Pub. No.: US 2019/0008810 A1
(43) Pub. Date: Jan. 10, 2019

(54) METHOD FOR PREVENTION, TREATMENT AND ALLEVIATION OF INFECTIOUS DISEASES AND DISORDERS

(71) Applicant: REPLICON HEALTH OY, Espoo (FI)

(72) Inventors: Petteri Hirvonen, Helsinki (FI); Peter Eriksson, Helsinki (FI); Risto Kaksonen, Espoo (FI)

(21) Appl. No.: 16/067,732

(22) PCT Filed: Jan. 4, 2017
(86) PCT No.: PCT/EP2017/050153

§ 371 Date: Jul. 2, 2018

Related U.S. Application Data
(60) Provisional application No. 62/274,432, filed on Jan. 4, 2016.
(60) Provisional application No. 62/274,446, filed on Jan. 4, 2016.

(51) Int. Cl. A61K 31/191; A61P 31/18; A23L 33/10; A61P 31/06; A61P 29/00; A61P 31/10; A61P 31/16; A61P 33/00; A61K 9/00

(52) U.S. Cl. CPC A61K 31/191; A61P 31/18; A23L 33/10; A61P 31/06; A61K 9/0053; A61P 31/10; A61P 31/16; A61P 33/00; A61P 29/00

(57) ABSTRACT

Provided are compositions for non-stressful activation of cellular, tissues specific and systemic immune defenses in the treatment of bacterial, virus, fungal or parasite (protozoa) infections and infections/inflammation arising from toxins or toxic agents, and related diseases and/or disorders. In particular, pharmaceutical compositions, dietary supplements and nutritive compositions comprising one or more compounds selected from the group consisting of D-glyceric acid, DL-glyceric acid and salts and esters thereof for use in treating, preventing or alleviating a communicable and/or infectious disease or disorder and/or related inflammation are described.

### FIELD OF THE INVENTION

The present invention relates to a composition comprising D-glyceric acid, DL-glyceric acid and/or their salts or esters for use in therapy, in particular in the alleviation, prevention and even healing of communicable and infectious diseases and disorders as well as inflammation related thereto. Furthermore, the present invention relates to the use of said composition for non-stressful activation of cellular aerobic energy metabolism and anti-inflammatory pathways to activate cellular, tissues specific and systemic immune defenses against bacterial, virus, fungal and/or parasite (protozoa) infections and infections/inflammation arising from pathogenic toxins or toxic agents, and related diseases and/or disorders. In addition, the present invention relates to said composition as a pharmaceutical composition, dietary supplement or nutritive composition.

### BACKGROUND OF THE INVENTION

The body of all vertebrates is under continuous attacks of various disease pathogens all the time. It is estimated that a normal human body faces 1 million attacks per day. Thus the fight against various disease pathogens is permanent and the immune system as a whole as well as cellular/tissue level defenses are more or less activated all the time. In healthy state this kind of a normal battle is called subclinical infection or better subclinical inflammation. In subclinical infection/inflammation the immune system can manage the situation and normal physiological balance is restored. When the immune defense or its control is somehow defeated clinical (level) infection starts with pro-inflammatory manifestations that initiate enforced counter reaction by cells of the innate and when needed also the adaptive immune system.

Furthermore, in pathological conditions, e.g. viral or bacterial infections, the immune systems activates into much higher degree compared to subclinical inflammation or normal physiological stress. Cytokine levels are increased dramatically and e.g. CRP (C-reactive protein) values can increase 100- or even 1000-fold compared to normal levels.

One solution to fight bacterial infections is the use of antibiotics. The problem with antibiotics is that they are not efficient against viral infections and furthermore bacteria can develop resistance against them. Similar problems arise in the use of other antimicrobial agents e.g. against protozoal infections. Especially increasing resistance is extremely big problem when using excessive amounts of antimicrobial agents. Another solution is to manage the systemic effects of inflammation and fight infections is the use of e.g. non-steroidal anti-inflammatory drugs (NSAIDs), corticosteroids (SAIDs), and other immunosuppressive agents that suppress inflammation. The problem with longer term use of immunosuppressive agents such as corticosteroids can be the adverse side effects like muscle weakness and bone loss. It is also important to notice that the primary cause of the disorder causing inflammation is typically not healed by these immunosuppressive agents. For the sake of Example cortisone (a synthetic glucocorticoid) suppresses elevated inflammation and inflammation response inter alia by inhibiting NF-kB (nuclear factor kappa-light-chain-enhancer of activated B cells) signaling through glucocorticoid receptor (GR). However, in acute infections the activation of NF-kB would typically be needed for the healing and resolution of the initial cause of inflammation. Traditional NSAIDs face the same problem as corticosteroids in that the underlying cause of the inflammation is not addressed, just the symptoms are alleviated. On positive side NSAID have in general less long term adverse side effects compared to SAIDs.

Thus, there still exists a need to provide improved means and methods that are effective in the treatment, prevention and/or alleviation of acute infections based on bacterial, virus, fungal or parasite (protozoa) disease pathogens and/or infections/inflammation based on some pathogenic toxins or toxic agents, and related diseases and/or disorders.

### SUMMARY AND DESCRIPTION OF THE INVENTION

The present invention relates to non-stressful and simultaneous activation of cellular aerobic energy metabolism and antioxidant and anti-inflammatory defenses by a composition comprising D-glyceric acid (“DGA” or “D-glycerate”), DL-glyceric acid (“DLGA”) and/or their salts or esters, together “the D-glycerate group” or “the DGA group”. (Non-stressful means “without excessive reactive oxygen species (ROS) generation”.) Based on experiments performed in accordance with the present invention, the efficacy of this activation is very consistent, rapid and surprisingly strong in wide range of cell and tissue types. Thus, it is evident that the administration of D-glycerate group activates major metabolic pathways in cells. As shown below, this non-stressful activation leads to significant enhancement of anti-pathogenic defenses in cells, target tissues and even in systemic defenses against communicable diseases and disorders.

Accordingly, in general the present invention relates to methods and compositions for use in methods of treating, preventing or alleviating a communicable and/or infectious disease or disorder and/or related inflammation, by non-stressful and simultaneous activation of cellular aerobic energy metabolism and antioxidant and anti-inflammatory defenses in a subject in need thereof. More specifically, the present invention relates to a composition, preferably pharmaceutical composition comprising one or more compounds selected from the group consisting of D-glyceric acid, DL-glyceric acid and salts and esters thereof for use in treating, preventing or alleviating a communicable and/or infectious disease or disorder and/or related inflammation.

Previously, D-glyceric acid (DGA) has been described to enhance alcohol metabolism; see, e.g., U.S. Pat. No. 7,666,909 ([4]). In related scientific article [5] it was reported that administration of ethanol and D-glyceric acid calcium salt to rats expedited the metabolism of alcohol. Habe et al. ([6]) showed in an in vitro study that D-glyceric acid can increase viability of ethanol-dosed gastric cells. Related to that article there is a patent application [7] that relates to alcohol induced gastrointestinal track mucous membrane damage and protection against it. WO 2006/112961 A2 ([8]) teaches the use of a composition comprising a tingling sensate and a food acid for the treatment of xerostomia (dry mouth), wherein the food acid present in the composition can be glyceric acid. In the

WO2015/036656A2 ([9]) the therapy of so called non-communicable diseases and disorders using DGA is described.

In contrast, based on the observations of the experiments described in the appended Examples, the present invention for the first time provides DGA, i.e. a composition comprising one or more compounds selected from the group consisting of D-glyceric acid, DL-glyceric acid and salts and esters thereof for use in the treatment of a communicable and/or infectious disease or disorder and/or related inflammation. As explained further below but without intending to be bound by theory it is believed that this effect is achieved through non-stressful (=without excessive ROS generation) and simultaneous activation of cellular aerobic energy metabolism and antioxidant and anti-inflammatory defenses in the subject in need. Thus, it is believed that this effect may be achieved by non-stressfully enforcing innate immune system, and by simultaneously and non-stressfully enforcing tissue specific and cellular anti-inflammatory, anti-microbial and cytoprotective defenses against bacterial, virus, fungal or parasite (protozoal) infections or other infections, e.g. based on some pathogenic/environmental toxins or toxic agents. In particular, it is believed that the effect of treating, preventing or alleviating viral infections and/or protozoal infections may be achieved by suppressing viral replication and protozoa oocyst shedding in host cells and tissues inter alia by up-regulating inducible heme oxygenase pathway (HO-1) activity.

Preferably, the composition is designed to be administered as a replacement for antibiotics, anti-microbial agents and/or anti-inflammatory substances, or in combination therapy with antibiotics, anti-microbial agents, anti-inflammatory substances and/or other effective molecules and/or preparations.

Typically, the disease or disorder is an infection and/or related inflammation in epithelial and/or endothelial cells or tissue, preferably wherein the epithelial cells or tissues comprise epithelium of the eyes, respiratory tract, reproductive tract, urinary and/or gastrointestinal tract.

In a preferred embodiment, the disease or disorder to be treated is a viral infection, protozoal, fungal and/or other infection, preferably wherein the disease or disorder is selected from the group consisting of seasonal flu, non-seasonal flu, viral influenza, ebola, rabies, hepatitis, HIV/AIDS, herpes, polio, meningitis, conjunctivitis, keratoconjunctivitis sicca, keratitis, lacrimal gland inflammation, gastroenteritis, diarrhea, constipation, inflammatory bowel disease, including Crohn's disease and ulcerative colitis, other inflammation based disorders like diverticulosis, tuberculosis, sepsis, haemophilus influenzae (bacterial) infection, antibiotic resistant bacterial infection (e.g. MRSA), salmonella, pneumonia, tetanus, and protozoa based infections like coccidiosis, toxoplasmosis and malaria. The composition may be in a form of a solution, syrup, powder, ointment, mixture, capsule, tablet, or an inhalable preparation, or wherein the composition further comprises a pharmaceutically acceptable excipient preferably the composition is in a form suitable for parenteral, oral, topical or inhalable administration and/or the composition is part of a beverage, a food product, a functional food, a dietary supplement, or a nutritive substance. The composition may be mixed with the feed thereby enhancing health of subjects in need and simultaneously e.g. improving feed conversation ratios in production animal industries; also morbidity and subsequent mortality may be reduced.

In a preferred embodiment the dose is 1-2×200 mg 2-4 times a day, in severe infection or inflammation preferably from 5 to 10 mg/kg body weight once, twice, three or four times a day.

The composition for use according to the invention may be for increasing the muscle yield per gram of nutrition, and preferably simultaneously decreasing fat content, and/or alternatively decreasing nutrition consumption without losing muscle mass; this is mostly due to reduced infections in gastrointestinal tract and thus enhanced feed intake.

The present invention further relates to a method of treating, preventing or alleviating a communicable and/or infectious disease or disorder and/or related inflammation in a subject in need thereof comprising administering an effective amount of one or more compounds selected from the group consisting of D-glyceric acid, DL-glyceric acid and salts and esters thereof to the subject, preferably by non-stressful (=without excessive ROS generation) and simultaneous activation of cellular aerobic energy metabolism and antioxidant and anti-inflammatory defenses in the subject

Hence, in view of the effect disclosed for DGA in context with infectious diseases in the human body and in tested animals for the first time as illustrated in the Examples, the present invention generally relates to a method of treating, preventing or alleviating a communicable and/or infectious disease or disorder and/or related inflammation, by non-stressful (=without excessive ROS generation) and simultaneous activation of cellular aerobic energy metabolism, and preferably antioxidant and anti-inflammatory defenses, in a subject in need thereof, e.g. by DGA or one or more compounds bringing substantially the same effect(s) including DGA precursors, prodrugs, derivatives, and the like. Furthermore, the term “effective amount” in accordance with the present invention means that said one or more compounds selected from the group consisting of D-glyceric acid, DL-glyceric acid and salts and esters thereof are present and used as the effective ingredient(s).

Preferably, either method of the present invention is for and accompanied with, respectively, activation of cellular, tissue specific and systemic immune defenses and their control against bacterial, virus, fungal or parasite (protozoa) infections and infections/inflammation arising from toxins or toxic agents, or a related disease and/or disorder.

In a preferred embodiment, the method of the present invention comprises administration of a composition comprising an effective amount of one or more compounds selected from the group consisting of D-glyceric acid, DL-glyceric acid and salts and esters thereof as described herein above and further below. In addition, or alternatively, the disease or disorder to be treated may be any disease or disorder described herein in context with composition of DGA for use according to the present invention.

In a further embodiment, the method of the present invention disclosed herein may be used for increasing the muscle yield per gram of nutrition, and preferably simultaneously decreasing fat content, and/or alternatively decreasing nutrition consumption without losing muscle mass in a subject in need thereof.

Unless otherwise specified, the terms, which are used in the specification and in the claims, have the meanings commonly used in the field of biochemistry, particularly in the field of inflammation and infectious diseases related studies.

The term “subject in need” refers to humans and animals. The composition of the present invention is useful for enhancing metabolism in subjects in need. The composition is suitable for use in humans. The composition is also suitable for animals. Communicable disease is an infectious/contagious disease communicable by contact with a subject who has it, with a bodily discharge of such a patient/subject, or with an object touched by such a subject/patient or by bodily discharges. Infection can be latent for a long time in the body without clinical manifestations. Communicable disease can be directly caused by pathogens like viruses, bacteria, protozoa and/or fungus or indirectly by pathogens, e.g. by mycotoxins, endotoxins, exotoxins and other similar environmental toxins.

Sustainable aerobic energy metabolism (sAEM) is defined as ATP generation (and ATP consumption), that does not lead to excessive proton pile up in the cytosol and/or excessive intracellular ROS formation, i.e. sAEM can balance itself and produce ATP at needed rate (continuously). Misfunctioning AEM is the opposite of sAEM. Prolonged infection due to dysfunction in energy metabolism can be caused by a defect in mitochondria itself or by a dysfunction in cooperation of mitochondria and other cell organelles.

NRF1 (nuclear respiratory factor 1) is an important transcription factor for sAEM. It activates the expression of some key metabolic genes regulating nuclear genes required for respiration (OXPHOS), heme biosynthesis, and mitochondrial DNA transcription and biogenesis. Nrf2/ARE is another extremely important transcription pathway for sAEM because it can via HO-1 and NRF1 promote mitochondrial biogenesis and independently reduce ROS generation. Third important transcription factor or coactivator for sAEM is PGC-1a (peroxisome proliferator-activated receptor gamma coactivator 1-alpha). PGC-1α is regulates the genes involved in energy metabolism. PGC-1α is a regulator of mitochondrial biogenesis and function. This protein can interact with, and regulate the activities of, cAMP response element-binding protein (CREB) and nuclear respiratory factors (NRFs). It provides a direct link between external physiological stimuli and the regulation of mitochondrial biogenesis. Furthermore, recent research has shown that PGC-1a can modulate lactate metabolism and prevent acidosis.

The transcriptional factors and master regulatory pathways that are simultaneously and non-stressfully activated in the invention are PGC-1a, NRF1 and Nrf2/ARE. Naturally in here, the term “simultaneous activation” does not mean that all hundreds of PGC-1a, NRF1 and Nrf2/ARE downstream pathways are activated; only that in minimum the most important pathways related to energy metabolism (e.g. OXPHOS and/or LDH) and anti-oxidant and anti-inflammatory defenses (like HO-1 pathway) are/can be activated to above normal level. For simplicity reasons, PGC-1a, NRF1 and Nrf2/ARE transcription factors/pathways are further defined together as the umbrella transcriptional pathways for regulating and promoting sustainable aerobic energy metabolism (later the “UTPfsAEM”), thus in the invention relevant parts of the UTPfsAEM are non-stressfully activated (SCHEME A and SCHEME B). This activation leads to claimed therapeutic effects as described in more details below. In the invention both efficient energy generation and its efficient use in enhanced.

Enhancement of mitochondrial oxidative phosphorylation (OXPHOS) is the main enhancer of aerobic energy metabolism but as seen below there are also other means that can enhance aerobic energy metabolism e.g. by generating cytosolic NAD+ and consuming protons, i.e. reducing acidosis (SCHEME C). For glycolytic cells that don't primarily use oxygen in their energy metabolism, enhancement of aerobic energy metabolism is defined as providing additional solutions for generating NAD+ for glycolysis and/or reduction of cytosolic proton concentration and acidosis. When aerobic energy metabolism functions sustainably cells can both generate and use ATP energy rapidly and for sufficient time e.g. for the resolution of an inflammatory attack. For the sake of clarity and completeness it is further defined that in the invention the activation of NRF1 covers, on top of complexes I-IV of the ETS (SCHEME C), also the activation of glycerol phosphate shuttle (GP-shtle) and ATP synthase (ATPase), and that the activation of PGC-1a and Nrf2/ARE modulate positively also the nuclear transcription of GRHPR and BVR genes (SCHEME C). (The logic of the latter (indirect effects) definitions is based on demand-supply—guided down—and/or upstream activations of GRHPR and BVR enzymes (more below).)

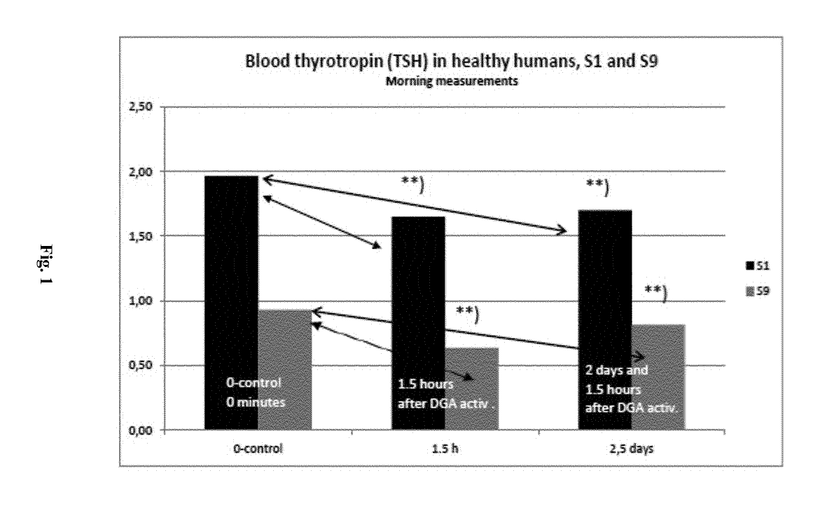

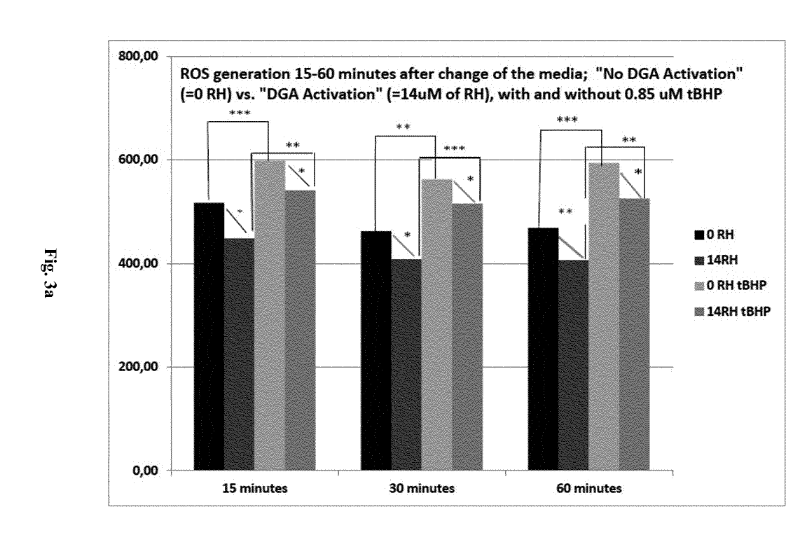

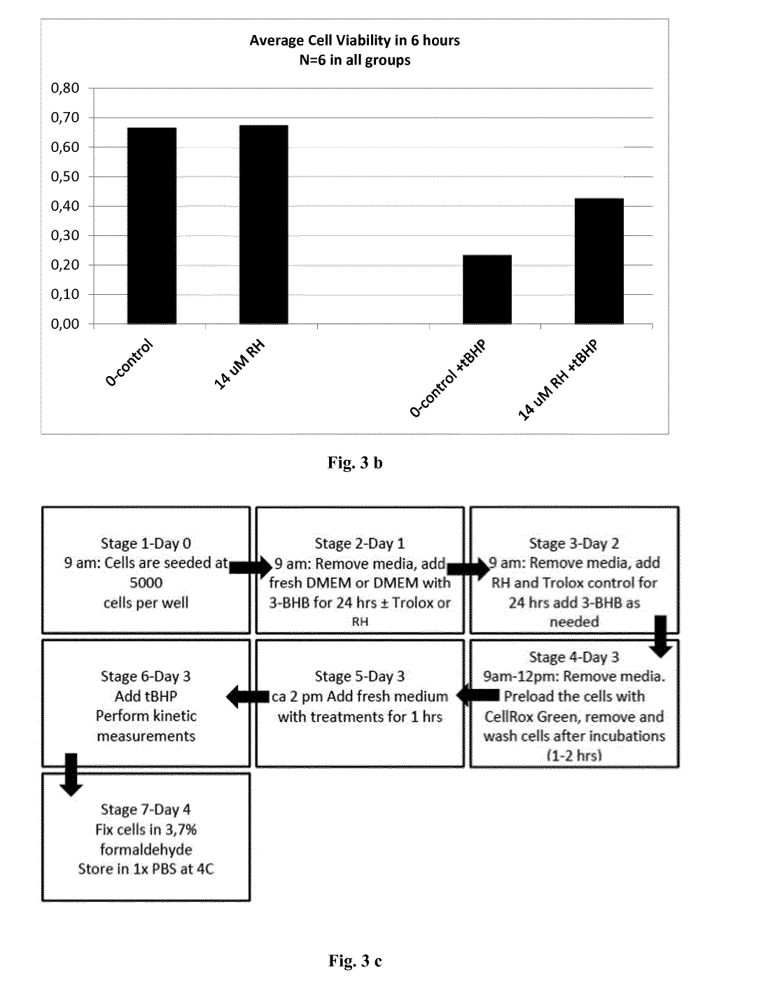

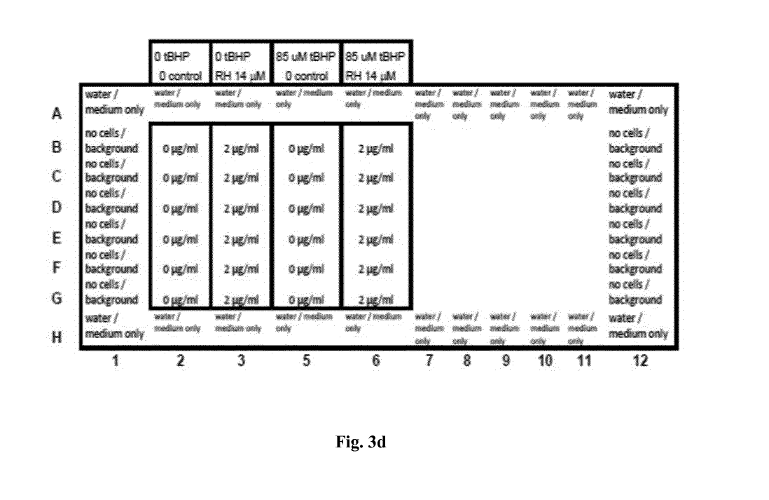

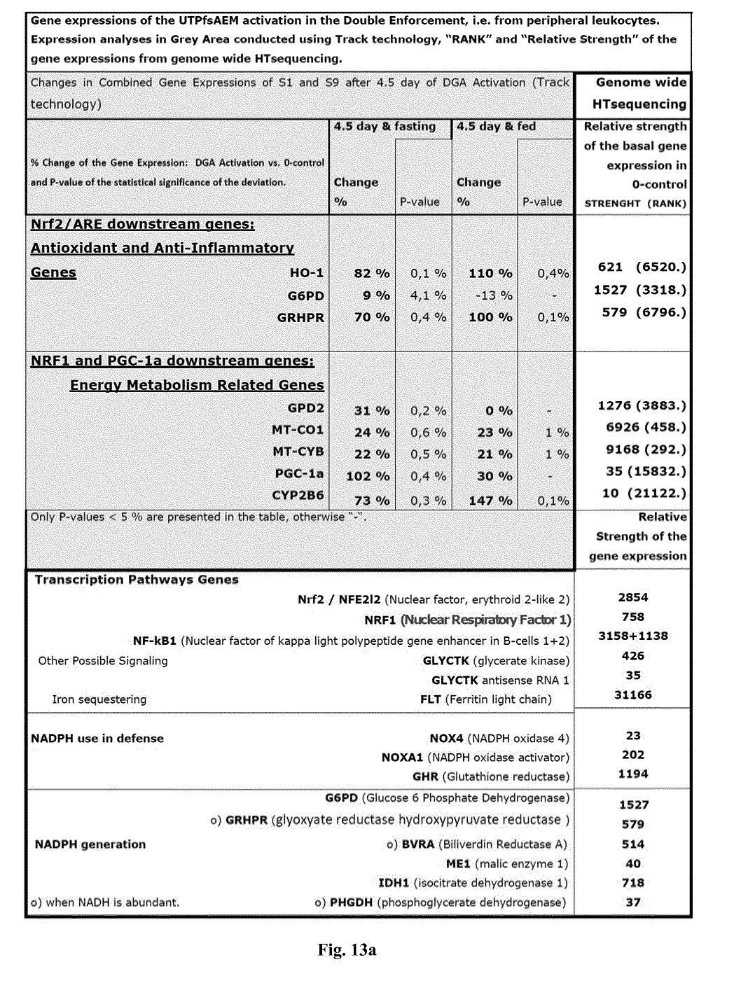

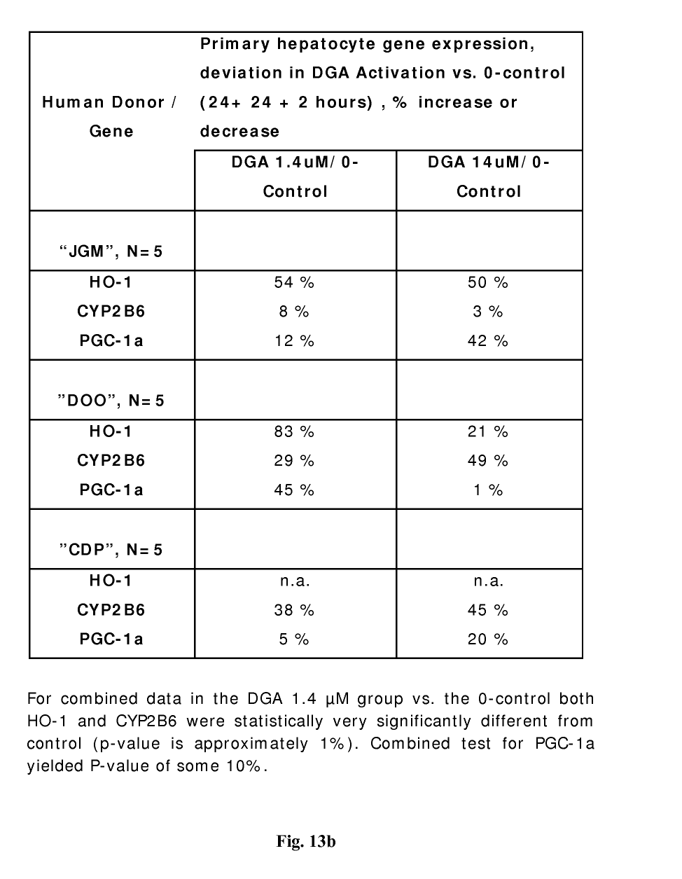

Using above definitions, the DGA administration can non-stressfully activate sAEM and UTPfsAEM. Systemic activation of energy metabolism is shown e.g. in FIG. 1 by rapid and sustained decline in thyrotropin stimulating hormone (TSH). Very briefly, reduced systemic need by TSH to keep cellular energy production at required homeostatic level is a clear sign of activation of cellular energy metabolism (ceteris paribus) after the D-glycerate administration compared to 0-control. Non-stressful activation of energy production is shown e.g. in Example 2. In Example 2 it is shown that the activation is non-stressful because D-glycerate can reduce net ROS generation in normal metabolic situation compared to 0-control. Importantly, it is further shown in Example 2 that aerobic energy production is rapidly activated (=ROS production temporarily increased) in induced disease model and despite that acute increase in ROS longer term net ROS generation is kept lower compared 0-control (FIG. 3a and FIG. 3c for the disease model induction). As explained in more detail in Example 2 indirectly shown is very fast activation of aerobic energy metabolism by the administration of D-glycerate in optic nerve astrocytes in induced extra stress. The non-stressful activation of sAEM and whole UTPfsAEM is also shown at gene expression level in FIGS. 13a and 13b, and explained in SCHEME A.

The DGA administration further leads to small accumulation of substrates for energy production. DGA can itself be converted into pyruvate via glycolysis even without need for cytosolic NAD+ and without net ATP consumption. This accumulation of energy substrates occurs also because actual ATP energy production is tightly regulated i.e. ATP energy is produced in the cells only for need, but the pathways providing substrates for energy production are not as strictly regulated. This kind of regulation leads to accumulation of energy “fuels”. In resulting situation there are 1) excess substrates for energy production, 2) aerobic energy production capacity of the cells is enhanced, and 3) UTPfsAEM related antioxidant and anti-inflammatory Nrf2/ARE (especially HO-1) pathway is clearly activated. This combination provided by the DGA group administration can promote systemic, tissue specific and intracellular anti-inflammatory and anti-infectious defenses clearly above normal physiological efficiency. SCHEME B describes some central pathways that are activated.

According to our Examples 1) PGC-1a, NRF1 and Nrf2/ARE master transcription pathways are induced at the same time, as well as also 2) aerobic energy metabolism, mitochondrial biogenesis and ROS scavenging (see example 1-5b, FIGS. 1, 2a and 2b, 3a, 6a and 6b and 13a and b). Thus, non-stressful activation of the umbrella transcriptional pathways for regulating and promoting sustainable aerobic energy metabolism (the “UTPfsAEM”) by the D-glycerate group has been shown. Because the non-stressful activation of UTPfsAEM is efficient in all tested active tissue types and both in aerobic and glycolytic cells it applies to wide range of cell types e.g. optic nerve astrocytes, peripheral leukocytes, hepatocytes, epithelial cells, myocytes, skeletal myotubes, erythrocytes, neurons and to other glial cells on top of astrocytes. As a follow-up, very wide range of tissue, and organ specific local improvements in defenses against pathological attacks materialize. Later this entirety is called as “the Local Enforcement”. Local Enforcement is extremely important for effective immune response and subsequent resolution of related inflammation. The Local Enforcement against pathological attacks is improved further by systemic effects because simultaneously the energy metabolism of the cells of the immune system is also non-stressfully activated. Later this non-stressful activation of the cells of the innate and the adaptive immune system is called as “the Double Enforcement”. “The Local Enforcement” combined with “the Double Enforcement” is called as “the DGA Activation” (SCHEME A).

At molecular level and in very simplified terms, the first solution of the DGA Activation for infectious diseases and disorders is to enhance ATP production and its efficient use by solving excessive cytosolic NADH and proton (H+) generation (SCHEME B). Simultaneous second solution provided by the invention is to enhance the indirect transporting of the energy of NADH molecules to NADPH molecules and/or NADP+ into NAD+ in the cytosol (SCHEME B). (NADPH molecules are vital components of anti-oxidant and anti-microbial, e.g. anti-bacterial, defenses, and NAD+ is vital for fast energy production.) Third simultaneous essential molecular level solution provided by the invention is the non-stressful activation of inducible heme oxygenase (HO-1) belonging to the UTPfsAEM. Non-stressful activation means without excessive increase in ROS generation (stress) and without excessive free iron formation. On top of cytoprotection, HO-1 activity has been shown to possess anti-viral activities. (Multistep heme degradation reaction also consumes several protons (H+) thus reducing acidosis.) Besides non-stressful DGA activation, HO-1 is induced also (somewhat stressfully) by endurance exercises.

All the above and below mentioned solutions are based on same technical feature, i.e. non-stressful activation of the UTPfsAEM. For schematic presentation on activated main downstream pathways see SCHEME B. For simplified presentation of the main metabolic fluxes see SCHEME C.

### DESCRIPTION OF THE EXPLANATORY DRAWINGS, SCHEMES A, B AND C

### Scheme A. Phases and Timeline Examples of the DGA Activation in Health and Disease

Scheme A (SCHEME A) summarizes major transcriptional pathways and their activation. See referred Examples in the graph for more precise information on therapeutic use and proof of the concept.

### Scheme A

Scheme B (SCHEME B) summarizes main downstream pathways that are activated. Sustaining optimal level of cytosolic NADPH by efficient reduction of NADP+ into NADPH is the main objective for the simultaneous enhancement of antioxidant, anti-inflammatory and anti-infectious defenses (Redox -ratio; NADPH/NADP+). Further, sustaining optimal level of cytosolic NAD+ by efficient use of chemical energy of NADH+ H+ is one main objective for enhancement of energy metabolism (Redox -ratio: NAD+/NADH).

Claimed actions presented in Schemes A and B are supported and proven by Examples 1-5b. Fast activation of the sAEM and subsequently the whole UTPfsAEM is inter alia shown in FIG. 1 and explained in its description below and in Example 5a. Very briefly, the fact, that the endocrinologic need of thyrotropin stimulating hormone (TSH) to keep the energy production at required homeostatic level is clearly reduced after the DGA Activation, shows (ceteris paribus) fast UTPfsAEM activation. TSH is very sensitive indicator and regulator of energy metabolism. Higher levels of TSH indicate that there is some shortage, e.g. dysfunction, in total energy production, and lower levels indicate that energy production works efficiently and is at good level.

Non-stressful activation of UTPfsAEM (=sAEM related parts of NRF1, PGC-1a and Nrf2/ARE transcription pathways) is shown by gene expression results in FIGS. 13a and 13b. Also, the long-term net ROS decline in stressed situation where net ROS generation is acutely increased (see Example 2) is a remarkable evidence of simultaneous activation of aerobic energy metabolism and antioxidant and anti-inflammatory defenses and their control.

Fast and non-stressful UTPfsAEM activation with elevated levels of substrates for energy production facilitates cellular and systemic anti-infectious defenses as seen in Examples 1a-1g.

Notably the DGA Activation can increase HO-1 expression significantly, i.e. by more than 100%, in peripheral leukocytes in vivo and some 50-80% increase in primary human hepatocytes in vitro as shown in Example 5b and FIGS. 13a and 13b. Endogenous carbon monoxide (CO), a product of HO-1 reaction, can ameliorate extremely wide range of acute inflammatory and infectious diseases. Endogenous CO has been reported to protect cellular structures in stressful conditions e.g. through binding to heme-moieties. CO can also ameliorate inflammation and subsequently reduce the expression of some cytokines, e.g. GM-CFS. In stressful conditions, like in acute ocular infections, and in their resolution the role of endogenous CO as a multipotent therapeutic molecule is increased, one reason being the reduction of oxidative and other damages to the tissues from the disease and/or disorder.

The experimental data from Examples 1a-1g, 2, 3, 4 and 5a-5b shows that the DGA Activation facilitates fast immune and inflammatory response. In transcriptional pathway terms this is the same as maintaining (on average) non-stressful activation of the UTPfsAEM and, when needed, accompanying that with temporary increase in ROS and activation of pro-inflammatory NF-kB transcription pathway.

Scheme C (SCHEME C) depicts major energy metabolic flows in a single cell. ATP producing mitochondrial electron transport system (“ETS”) located at the IMM (inner mitochondrial membrane) is the most important pathway that is activated by the DGA Activation. (For simplicity reasons, e.g. the outer mitochondrial membrane has been left out from the graph.) ETS creates most of the reactive oxygen species (“ROS”) in the cell. Glycerol phosphate shuttle (“GPshtle”) is part of the ETS and its activation is important for fast cytosolic NAD+ supply and buffering of the excess protons. Malate-aspartate-shuttles (MA-shtle) provide most of the needed cytosolic NAD+ but only indirectly. Also, cytosolic enzyme loops can assist in balancing cytosolic redox -states (see more below). TCA stands for tricarboxylic acid cycle, also known as the citric acid cycle.

Molecular level actions presented in SCHEME C (main parts explained below) are based on the evidence of multiple substrate level results from blood samples in vivo, in vitro ROS and viability analyses, and additionally on in vivo and in vitro gene expression studies from samples of human peripheral leukocytes and hepatocytes. Results are from whole physiological system (in vivo) and from four different tissue types (in vitro) from four different vertebrates. Presented molecular level flows, especially substrates for reduction - oxidation - reactions (“redox” -reactions) and excess proton buffering, are extremely important because especially in stressful events like infections and other pathological conditions the efficient resolution calls for continuous and sufficient supply of energy.

In Scheme C signals for “More ATP” are indicated by stars in the extra cellular fluid and inside of the cell. In the present invention, the main candidate creating the “signal” for increased energy metabolism in all active tissues is the increase in the concentration of the DGA in the body (see the star in extra cellular fluid in SCHEME C). Parallel activation of GLYCTK enzyme in the main metabolic direction of DGA can also facilitate such “signal” (see the star inside the cell in SCHEME C). Brief explanation, high and prolonged ATP demand, like seen in e.g. endurance exercise, can lead to overflow in the glycolysis pathway. This substrate overflow leads to build up of also 3- and 2-phosphoglycerates (see “3-P-G”) and subsequent endogenous increase of DGA by GLYCTK. In this explanation, signal “More ATP” and endogenous increase in DGA concentration coincides. Another parallel explanation is that high and prolonged ATP demand eventually turns GLYCTK gene pathway towards producing endogenous DGA and (“exceptional”) ATP. This direction can yield ATP and simultaneously it increases D-glycerate (DGA) concentration. From the cells, extra DGA will be transported into extracellular fluid through monocarboxylate transporters (see “MCT” in SCHEME C). The increase in endogenous DGA can thus have at least paracrine and even endocrinal signaling effects. Our Examples clearly show that administered exogenous DGA clearly has a strong effect in activating energy metabolism.

For our patenting purposes, it is sufficient that it has been shown that the activation of aerobic energy metabolism and related UTPfsAEM materializes fast/efficiently, non-stressfully, and notably with relatively low doses of D-glyceric acid both in vivo and in vitro.

In normal physiological conditions the cytosolic NAD+/NADH -ratio is extremely high (some reports say that it is even clearly above 100) and NADHP/NADP+ -ratio is clearly higher than 1. Extremely high NAD+/NADH -ratio is needed because of the vast and continuous metabolic flow through of the glycolysis. At the same time the existence of lactate related acidosis is a very clear proof that in stressful situation sufficient NAD+ pool can run out quickly. Because of the DGA Activation cytosolic NAD+/NADH and NADPH/NADP+ balancing can work more efficiently.

Enhanced supply of cytosolic redox substrates NAD+ and NADPH and increased buffering capacity of excess cytosolic protons facilitate energy production. Solution that they can provide is faster and non-stressful supply and use of cellular ATP both for the Local and for the Double Enforcements. Especially in stressful situations aerobic energy metabolism must be enhanced by efficient substrate shuttling between glycolysis (in the cytosol), the citric acid cycle and the ETS (in the mitochondrial matrix) because direct cytosolic ATP production needs it. It should be noted that ATP produced in the cytosol is more readily in use compared to ATP produced by ATP synthase complex into the mitochondrial matrix. Glycolysis can generate a lot of ATP fast if there is enough of NAD+ available. On the other hand, excess cytosolic H+ leads to so called acidosis and subsequent decrease in fast ATP production and reduced possibilities for ATP consumption. E.g. lactate dehydrogenase (LDH) enzyme ameliorates acidosis by converting NADH+ H+ into NAD+ (and pyruvate (PYR) into lactate). When ATP must be produced rapidly, e.g. by the immune system cells in acute infection, NADH and H+ molecules can “pile up” in the cytosol because the capacity of the mitochondrial transportation shuttles of NADH and H+ into the ETS is exceeded. Below it is further shown that there are even additional, previously not described, repeatable channels for continuous, sufficient and fast supply of NADPH and ATP for efficient and timely immune response. This novel proposal can be an additional explanation why HO-1 pathway activation possesses such tremendously wide therapeutic effects.

Requirements for enzymes that can form repeatable enzyme loops supporting efficient immune response are that they can function on both NADH and NADPH co-enzymes, and the catalyzed reactions are reversible, i.e. they function efficiently to both directions. Biliverdin reductase (BVR) can be such an enzyme. (In Prior art, only NADPH molecule has been described as the possible reduced acceptor but based on our research of public enzyme libraries BVR can use both NADH and NADPH.) Additionally, it has been clearly shown that the DGA Activation activates HO-1 pathway that yields biliverdin, a substrate for BVR. Furthermore, HO-1 pathway is active in extremely many cells types and it is important especially in circulating peripheral leukocytes that “gain” hemoglobin molecules from degrading red blood cells.

BVR reduces biliverdin (BV) into bilirubin (BR), and in that reaction, it can use the energy from NADH+ H+ molecules. When energy demand is high, cytosolic NADH+H+ is abundant as well as NADP+. The latter materializes because NADPH+ H+ is consumed in e.g. glutathione activation to be used against excessive oxidative stress (“ROS”) from mitochondria (SCHEME C). In these conditions, formed BR can be (more often than in normal conditions) converted back to BV and simultaneously convert NADP+ into NADPH+ H+. (It should be noted that cytosolic H+ (proton) concentration is reduced by these loops only if/when NADPH+H+ is consumed for neutralizing of free radicals.)

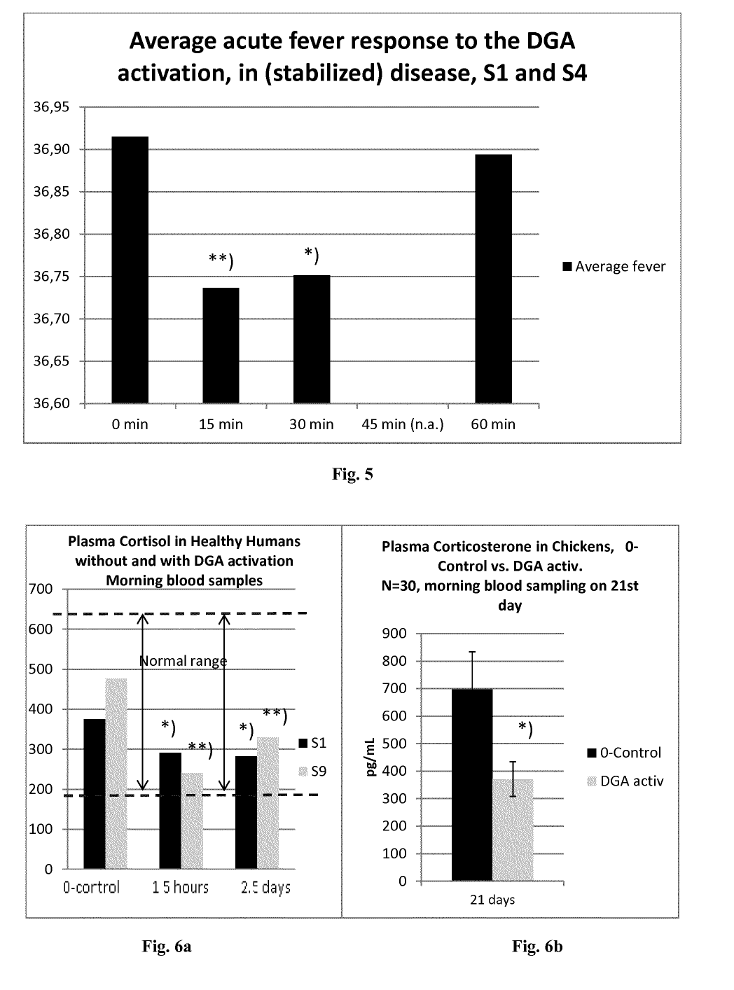

These kinds of enzyme loops can facilitate restoring redox -balance when ATP demand is high. Excess NADH is converted into NAD+ and excess NADP+ into NADPH. Formed NADPH can be used in ROS scavenging but also in important lipid and cholesterol (mevalonate pathway) related synthesis (not shown in SCHEME C). From 3-5 week feeding study with broiler chickens (see Example 5a and FIG. 6b) it can be concluded that fat formation is not increased (N=144), which shows that cytosolic NADPH supply for fatty acid synthesis does not increase in the DGA Activation more than is needed for normal homeostasis.

### BRIEF DESCRIPTION OF THE EXPERIMENTAL DRAWINGS

In all Figures “*” means that the p-value in statistical Students t-test of that particular measurement compared to relevant 0-control is less or equal than 5% (0,05). Mark “**” means that the p-value is less than 1% (p<0,01) and mark “***” is the same as p<0.001. All tests are one sided.

In the following, “DGAcs” is the calcium salt formulation of D-glyceric acid containing also water molecules (=D-glyceric acid calcium salt dihydrate). In aqueous solutions, it is dissolved into conjugate base of D-glyceric acid and calcium (and water).

FIG. 1: The Acute and Persistent Effect of the DGA Activation on the Energy Metabolism in Healthy Humans. Thyroid stimulating hormone (TSH) measured from standard EDTA blood sample. Morning measurements in “half fasting” state, i.e. half of the normal breakfast eaten 1.5 hours before the collection of the blood sample.

As can be seen from FIG. 1, the blood levels of TSH in S1 and S9 decline already in 1.5 hours compared to the 0-control after the DGA Activation, both after first administration as well as after 2 days of administration. This decline is very clear and statistically very significant. TSH regulates energy metabolism indirectly through the regulation of thyroid gland and thyroid hormones (T4 and T3, see Endocrinologic Proof of Concept and Example 5a and FIG. 6 for more info). Additionally, in literature it has been shown that the blood level of TSH has direct impact on mitochondrial activation and biogenesis in vertebrates. Based on these results of FIG. 1 combined with clear cut gene expression results in FIG. 13 it can be concluded that the Local Activation can enhance intracellular sAEM in vivo because of lower TSH level imply lower systemic stimulation need.

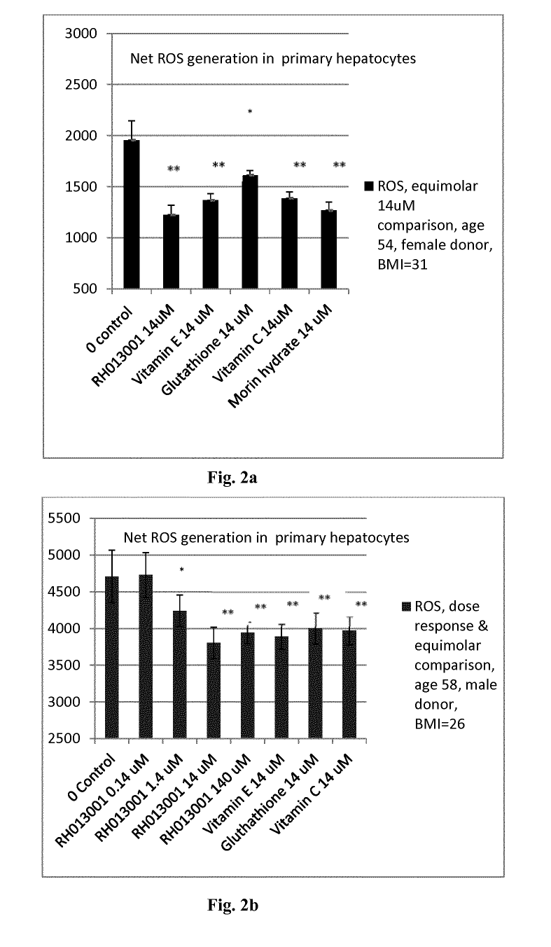

FIG. 2: In vitro demonstrations of effective ROS scavenging by the DGA activation in human primary hepatocytes, the dose dependence, and equimolar comparison to other efficient antioxidants. D-glycerate is coded as RH013001 in the graph. FIGS. 2a and 2b shows that antioxidant response elements (AREs) are clearly activated in these primary human hepatocytes. ARE -activation happens when Nrf2 detaches from cytosolic KEAP1 enzyme and moves to nucleus (SCHEME B).

FIG. 3a: The Longer Term and Acute Effect of the DGA Activation on the ROS Generation in Rat Optic Nerve Astrocytes, in health (0 μM tBHP) and in disease (85 μM tBHP). The DGA activation is the same as administering 14 μM of DGAcs (=14 μM of RH) in FIGS. 2a and 2b.

FIG. 3a shows that antioxidant response elements (AREs) are clearly activated in these primary rat optic nerve astrocytes in normal conditions and furthermore in induced extra stress (bolus tBHP addition).

Remarkable in FIG. 3a is also that calculated net ROS generation is temporarily increased more in the DGA Activation group compared to 0-control immediately after the bolus addition of tBHP.

FIG. 3b: The Longer Term and Acute Effect of the DGA Activation on the Cell Viability of Rat Optic Nerve Astrocytes, in health (0 μM tBHP) and in disease (85 μM tBHP).

FIG. 3c: Phases/stages of one of the Experiments with Rat Optic Nerve Astrocytes. In this experiment (Example 2) the disease model is generated by tBHP administration (see “Stage 6-Day 3”).

FIG. 3d: Rough plate Set Up of one of the Experiments with Rat Optic Nerve Astrocytes

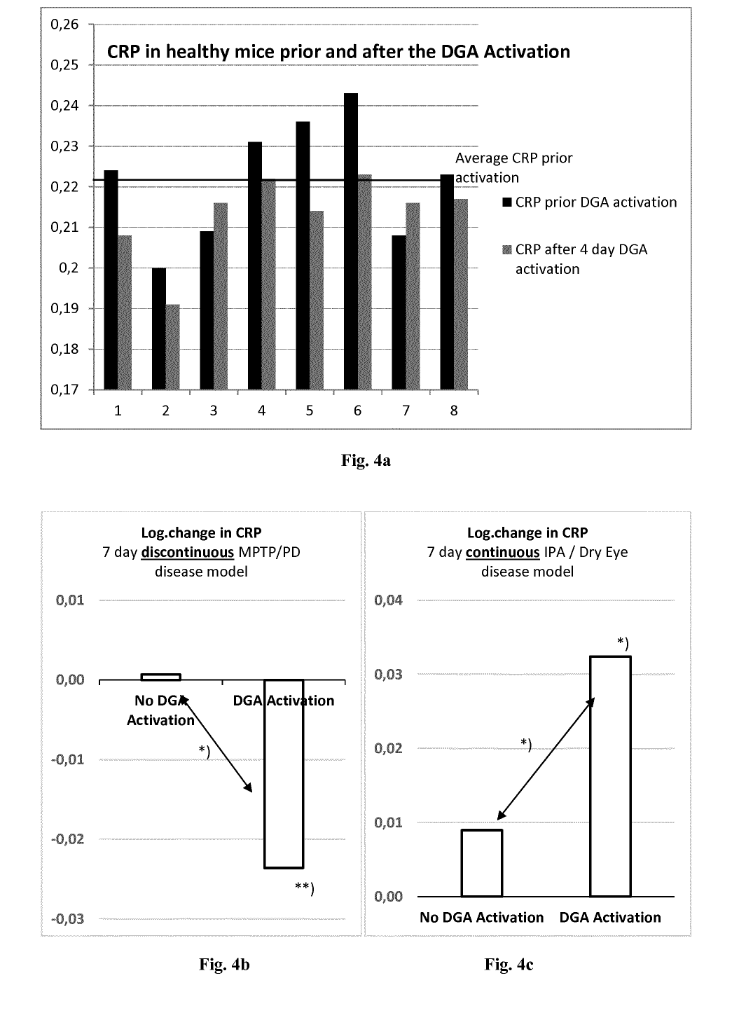

FIG. 4a: CRP in Health. 4-day DGA Activation's effect on blood CRP in healthy mice (N=8). Comparison is made to the same individual without the DGA administration, i.e. prior starting the DGA administration. In pairwise test the reduction of the CRP was significant (P=0.04). The tendency towards reduced CRP is because typical mice had higher initial CRP due to normal subclinical inflammation prior starting the administration.

FIGS. 4b and 4c: CRP in Two Different Disease Models, continuous vs. discontinuous. In FIG. 4b, results from discontinuous MPTP/PD disease model, and in FIG. 4c, results from continuous IPA/Dry Eye disease model. Logarithmic change of blood CRP during the 7-day disease follow up period of each mouse in all groups was calculated. In both models, there was a 4-day priming with 50% DGA dose before activating the disease model.

As can be seen from FIG. 4b, in discontinuous disease model CRP declined statistically significantly in the DGA Activation compared to 0-control with no

DGA Activation (p-value=0.015). This indicates healing from Parkinson's/MPTP Disease model. Notably in this discontinuous disease model the CRP decline was statistically even more significant when the CRP change in individual mice was compared (pairwise test, p-value=0005).

FIG. 4c shows clearly that the DGA Activation can, in continuous disease model, enhance inflammatory response. In IPA model CRP increased statistically significantly in the DGA Activation compared to 0-control with no DGA Activation (p-value=0.027). In simplified terms cytokines (especially IL-6) continue to be elevated causing the liver to keep producing CRP more proteins compared to 0-controls. More in Example 3.

FIG. 5: The Acute Average Effect of the DGA Activation on the fever in two humans in disease. See Example 4 and the description of the invention for more information on this remarkable ability of the DGA Activation to reduce fever mildly but very fast. (Original measurement results are presented in a table in FIG. 11.)

FIG. 6a: The acute (1.5 hours) and persistent (2.5 days) effect of the DGA Activation on the energy metabolism and metabolic stress/subclinical inflammation in healthy humans. Cortisol, endogenous human glucocorticoid, is measured from standard EDTA blood sample of two healthy humans. Scale is nmol/l (nano moles/litre). Morning measurements in “half fasting” state, i.e. half of the normal breakfast eaten 1.5 hours before the collection of the blood sample. See also FIG. 1, Example 5a and the description of the invention for more information on this remarkable ability of the DGA Activation to reduce cortisol in health. Cortisol is the stress hormone of the body and its role is also to reduce inflammation (an endogenous steroidal anti-inflammatory agent). These results show that the DGA Activation can reduce stress and subclinical inflammation.

FIG. 6b: FIG. 6b shows average blood corticosteroid (corticosterone) in 30 healthy broiler chickens without (N=20) and with (N=10) DGA Activation. The birds in both groups have been grown for 3 weeks exactly identically except that the DGA Activation group has received 9 mg/kg of body weight/day of DGAcs mixed into the standard broiler chicken feed. Remarkably the level of corticosterone in broilers declines statistically significantly (p-value=0.021) some 40%, i.e. similarly compared to humans in shorter test (see FIG. 6a). In FIG. 6b the standard error of the mean (SEM) for both groups is presented by error bars. Additionally, the gap between 0-control and the DGA Activation group remained or in fact widened at 28-day measurement point. (This data is not shown in the graph because in the 0-Control group one bird increased the average very heavily. Even without that observation the gap between the groups widened somewhat.) Also, lower average IL-6 levels were observed in broiler chickens after 21-28 days of DGA Activation compared to 0-control (for more information see the end of Example 5a).

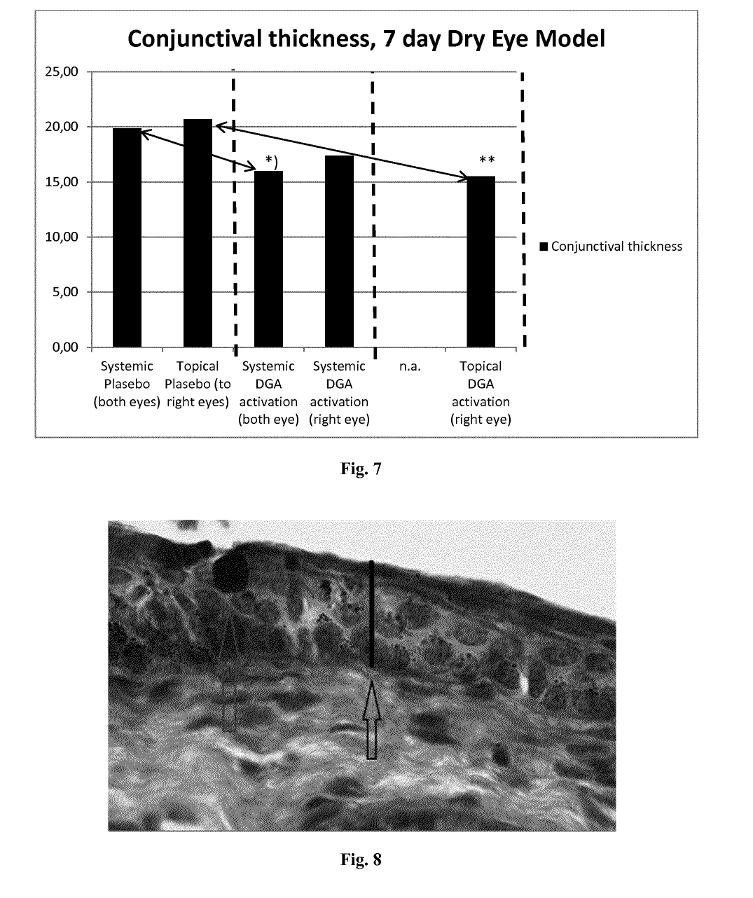

FIG. 7: The 7-day Effect of the DGA Activation on the Conjunctival Thickness. In this disease model, reduced thickness is a sign of improved epithelial cell layer health. In the experiment, there were both topical DGA Activation with placebo eye drops and systemic DGA Activation with normal chow tested against the 0-controls (=placebo eye drops or normal chow). As can be seen from FIG. 7 both topical and systemic DGA Activation can reduce the thickness of epidermal layer. For more information see Example 1 and the description of the invention.

FIG. 8: The 7-day exposure of mice to dry environment causes a thickening of the epidermis (black arrow pointing to the epidermal layer). Goblet cell (lighter arrow) density is an important parameter that reflects the overall health status of the ocular surface. These cells synthesize, store, and secrete large gel-forming mucins that lubricate and protect ocular surface from dryness. FIG. 8 shows an example of conjuctival epidermis and goblet cells.

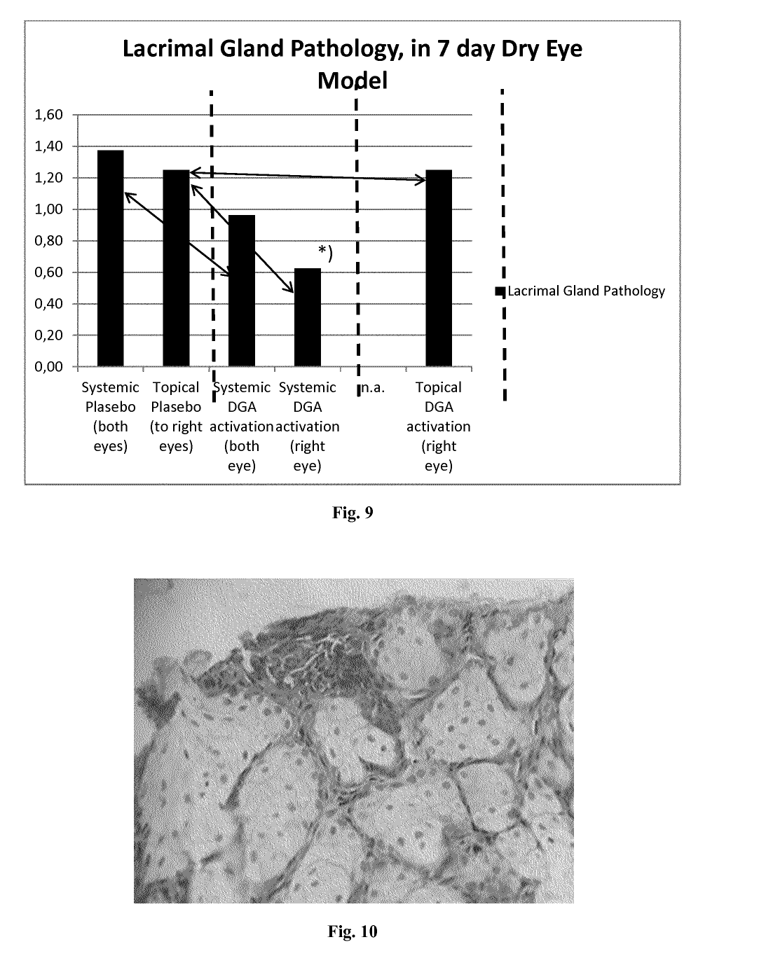

FIG. 9: The 7-day Effect of the DGA Activation on the Lacrimal Gland Pathology. Grading: 0=no visible change, 1=mild accumulation of mononuclear cells within the interstitium, 2=focal accumulation of mononuclear cells without any parenchymal destruction, 3=focal accumulation of mononuclear cells with parenchymal destruction, 4=extensive infiltration of mononuclear cells with severe tissue damage.

As can be seen from FIG. 9 only systemic DGA Activation can reduce lacrimal gland pathology. The protective effect compared to 0-control was statistically significant only in the eyes that were treated (=stressed) additionally with eye drops four times a day for 7 days. For more information see Example 1 and the description of the invention.

FIG. 10: The 7-day exposure of mice to dry environment initiates lacrimal gland inflammation. FIG. 10 shows typical Examples of inflammatory lesions found from the samples (grade 1).

FIG. 11: Numerical values of short term reduction in fever in disease after the DGA Activation (administration approximately 300 mg of D-glyceric acid mixed in water as calcium salt (=2× 200 mg of DGAcs). The results in this table are presented graphically in FIG. 5 and explained in Example 4. Additional Notes: 300 mg of D-glyceric acid is more precisely the same as 430 mg of calcium salt dehydrate formulation, because one calcium atom and 2 water molecules make up some 30% of the total molecular weight. *) Reduction in fever materialized but the symptoms of the disease e.g. pain remained. **) Rather stressful “absent day from work arrangements” by e-mail between 0 minutes and 15 minutes' measurements could possibly have kept fever up.

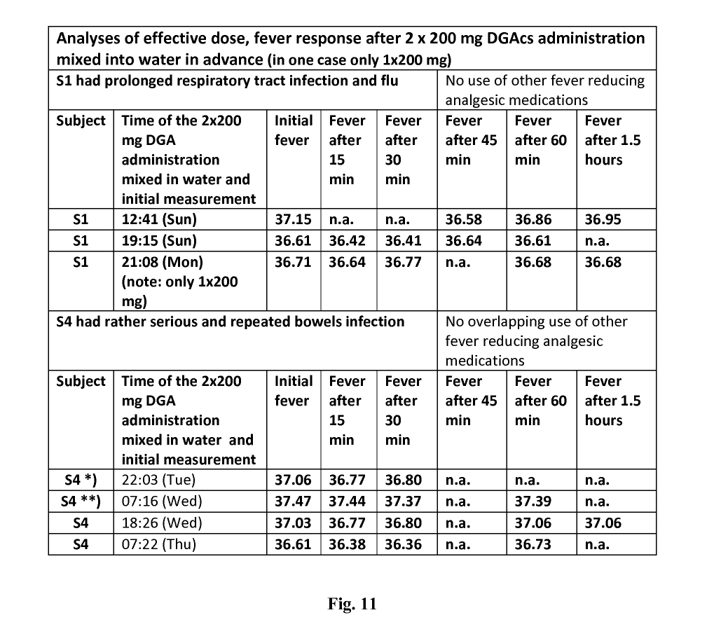

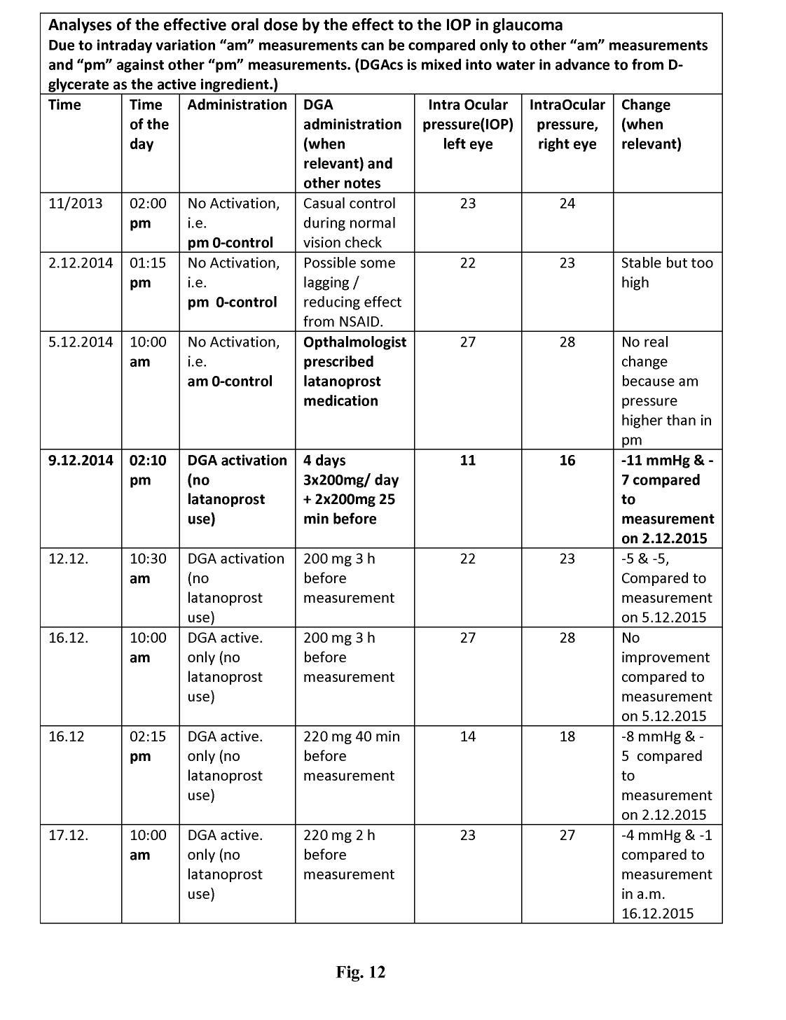

FIG. 12: Numerical values of the reduction in IOP in Glaucoma after DGA Administration. From data in the table in FIG. 12 one can observe that the DGA Activation seems to reduce intraocular pressure (IOP) but used acute dose 1*200 mg was not necessarily sufficient. Only in first measurement under the “full” DGA Activation (9.12.2015) there was very significant reduction in IOP compared to relevant control measurement (on 2^{nd} of Dec, 2015).

FIG. 13a: In this 4.5-day experiment samples of peripheral leukocytes were collected from standard blood sample of S1 and S9 in fasting condition (0 h), and 1 hour (1 h) after taking 75 grams of glucose (Glutole, Biofile Pharma, 330 ml) for glucose tolerance test. After the collection of the blood samples the leukocytes were immediately separated, and after separation immediately lysed by stop solution and stored in freezer in line with the instructions by the service provider.

Gene expression was measured from those peripheral leukocyte (=white blood cells). Additionally, basal (=0-Control) gene expression of all genes was later measured for S1. This was done using a different gene expression method for genome wide sequencing.

The doses used were 250 mg taken twice a day, in the morning and before going to bed. The last DGA dose was double in size (some 8 mg/kg), and it was taken in the same morning as the collection of blood samples. Relatively high dose and 2.5 hours to measurement was chosen to see clearer dose response in gene expression from peripheral leukocytes.

As can be seen from FIG. 13a the systemic DGA Activation can activate NRF1, PGC-1a and Nrf2/ARE gene expression very clearly in vivo. For further information on these remarkable results see Example 5 b and the description of the invention.

FIG. 13b: Gene Expression results from Primary Hepatocytes after 48 h+2 h of the DGA Activation vs. 0-control. For combined data in the DGA 1.4 μM group vs. the 0-control both HO-1 and CYP2B6 were statistically very significantly different from control (p-value is approximately 1%). Combined test for PGC-1a yielded P-value of some 10%. Primary human hepatocytes were purchased from Celsis In Vitro Technologies (1450 South Rolling Road Baltimore, Md. 21227, USA). Primary hepatocytes were received from three different post mortem human donators. JGM is a female donor (age 54). JGM's net ROS generation results are presented in FIG. 2a. DOO is a male donor (57 years). CDP is a male donor (58 years). CDP's net ROS generation results are presented in FIG. 2b. AS can be seen from graphs 2a and 2b net ROS generation declined for both JGM and CDP statistically very significantly after DGA Activation. This is also the case for DOO.

In this experimental model the cells in the 0-Control and in the two DGA Activation groups were normally and identically incubated before DGA Activation. Incubation the medium is changed every 24 hours. When the 48 hour+2 hour DGA Activation started, the only difference was the addition of either 1.4 μM or 14 μM of DGA to the used medium. The D-glyceric acid calcium salt was carefully mixed in to medium before the administration. Thus, it was separated from the calcium salt.

For combined data in the DGA 1.4 μM group vs. the control both HO-1 and CYP2B6 were statistically significantly different from control (P-value is approximately 1%, i.e. the result was statistically very significant). Combined test for PGC-1a yielded P-value of some 10%, which indicates that also PGC-1a was activated compared to control. Importantly the deviations from relevant controls are perfectly in line with in vivo results from leukocytes.

It can be concluded that there is a clear tendency for inducible heme oxygenase (HO-1) to rise in hepatocytes after the use of the DGAcs which is a clear indication of Nrf2/ARE activation. Furthermore, the expression of the master regulatory gene of energy metabolism, PGC-1a rises also. PGC-1a activation is backed up by CYP2B6 activation because its activation is induced by PGC-1 a.

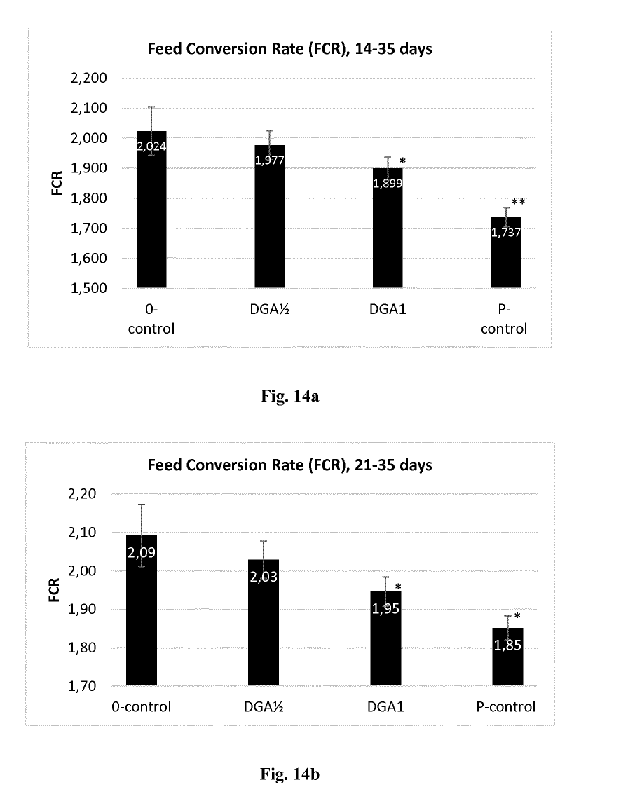

FIGS. 14a, 14b and 14c: Feed conversion rates (FCR) after provided protozoa infection (=coccidial challenge) in broiler chickens. Briefly, eimeria mix was given at day 15. Relevant periods to observe differences in weekly FCRs between treatment groups are thus 14-35 days (FIG. 14a), 21-35 days (FIGS. 14b) and 28-35 days FIG. 14c). See Example 1 g for more information of the experimental setting and interpretation of the results.

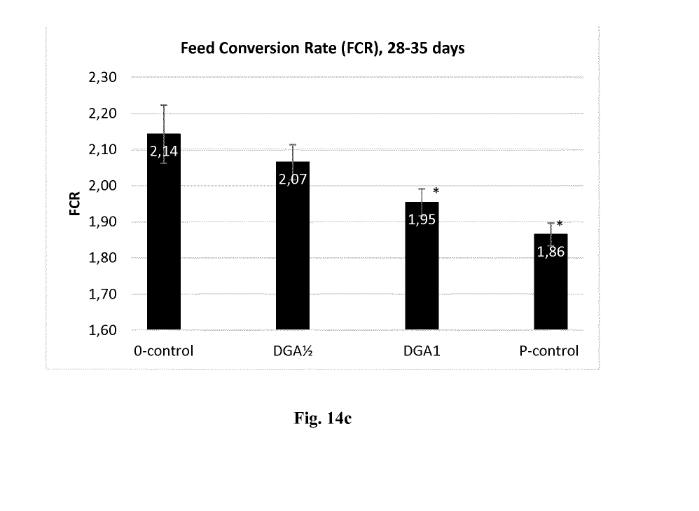

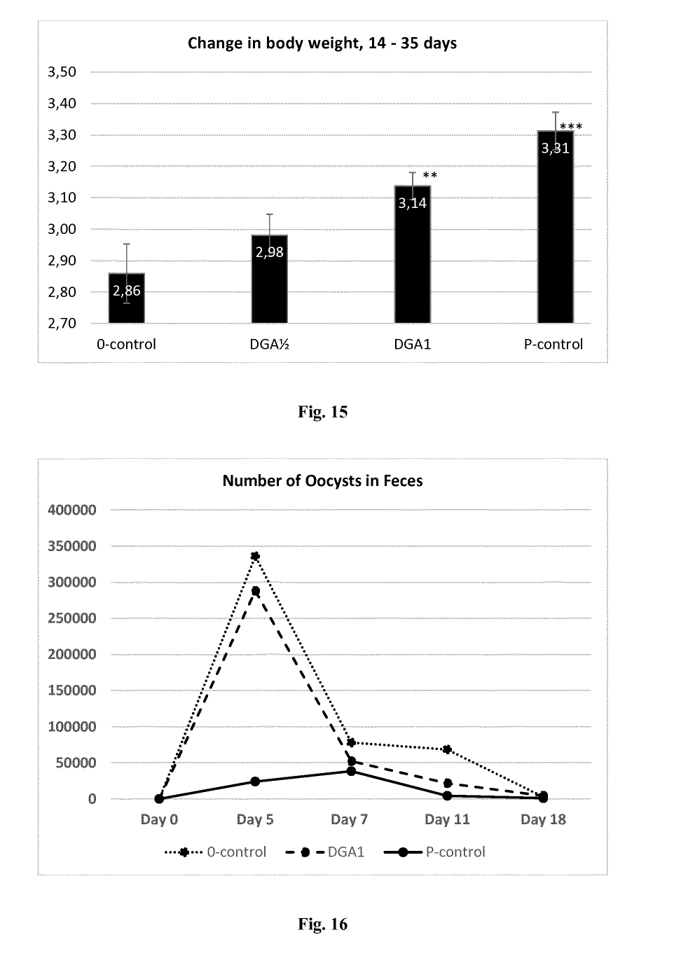

FIG. 15: Growth after coccidial challenge, days 14-35. More info in Example 1g.

FIG. 16: Difference in oocysts shedding (replication) after coccidial challenge. Oocysts sample are taken on 5 different days. One just before the challenge and the other ones at selected relevant days after the challenge. This analyses and its results are very important in showing that the DGA Activation can reduce pathogen replication in both DGA1 and DGA½ groups compared to 0-control group statisticalle significantly, for more info see Example 1g.

### DETAILED DESCRIPTION OF THE INVENTION

The core of the present invention is that cellular aerobic energy metabolism is non-stressfully activated by administrating a composition comprising D-glyceric acid, DL-glyceric acid and/or salt or ester thereof. This is equivalent to fast, simultaneous and non-stressful activation of PGC-1a, NRF1 and Nrf2/ARE transcription pathways (the UTPfsAEM). As described in chapter Summary of the Invention this DGA Activation leads to multiple positive effects in enhancing immune defenses and thus to the prevention, alleviation, and even resolution of wide range of communicable and infectious diseases.

D-glyceric acid is a weak acidic compound. It can be prepared e.g. by oxidation of glycerol. D-glyceric acid can be liberated from its commercially available calcium salt form by simple treatment with dilute hydrochloric acid. Salt formulations are also water soluble. In aqueous solutions, salt formulation is dissolved into conjugate base of D-glyceric acid and calcium (and water). In used extremely small relative concentrations, the conjugate bases can attract protons from water molecules and form D-glyceric acid without having any meaningful effect on the acidity of target tissues or extracellular fluids of the subject in need. Furthermore, the amounts of calcium are negligible compared to physiological concentrations and use of calcium.

Being an organic acid, DGA is also capable of forming esters. DGA can be liberated from its esters, for instance, by esterase enzymes. E.g. in the human body, these enzymes are present in the wall of small intestine where they split esterified nutrients into a form that can be adsorbed from the digestive tract. DGA is typically not directly involved in the normal growth, development or reproduction of an adult organism. Unlike its phosphorylated forms (phosphoglycerates) DGA is not produced in bigger amounts during normal sugar catabolism in the human body. Only very small amounts of DGA have been found in the body [10].

L-glyceric acid is biologically inactive enantiomer but it can be in small amounts converted into DGA via hydroxypyruvic acid (HPA). That is one reason why racemic DL-glyceric acid, containing 50% of active DGA enantiomer may be used in accordance with the present invention. In this context, it should be understood that instead of directly using DGA or a salt or ester thereof in accordance with the present invention equivalent compounds may be used which for example are converted to DGA such as HPA or induce or enhance the production of endogenous DGA in the human body and bring about substantially the same effect. However, HPA is less preferred because the acute doses needed are typically somewhat higher in communicable diseases than in non-communicable diseases and because it is generally known that HPA can be toxic in very high doses. Nevertheless, when HPA (or whatever other substance as well) is administered in such a way that it can only or mainly be converted into DGA in the subject in need thereof, its use is encompassed by the teaching of the present invention.

A composition which is useful in the present invention comprises one or more compounds selected from D-glyceric acid, DL-glyceric acid and/or salts and esters thereof. Said compounds are for use in a method of enhancing direct and indirect mitochondrial metabolism. Said compounds or a composition comprising one or more of said compounds are also for use in a method of treating, preventing or alleviating a communicable and/or infectious disease or disorder and/or related inflammation.

The present invention is useful in treating, preventing or alleviating a communicable and/or infectious disease or disorder by simultaneous enhancement of the immune systems and tissue specific and cellular antioxidant and inflammatory defenses against disease pathogens like microbial infections (bacterial, virus, fungal and/or parasite (protozoa)) or infections/inflammation based on some pathogenic/environmental toxins or toxic agents.

The present invention is useful in the therapy areas selected from the following non-limiting groups.
1) All bacterial infections in vertebrates that the innate and/or the adaptive immune system can fight against, including but not limited to tuberculosis, sepsis, Haemophilus influenza bacterial infection, antibiotic resistant bacteria infection (e.g. MRSA), salmonella, pneumonia and tetanus.
2) All viral infections in vertebrates that the innate and/or the adaptive immune system can fight against, including but not limited to seasonal flu, various types of virus influenza, ebola, rabies, hepatitis, HIV/AIDS, herpes, polio and meningitis.
3) All other infections in vertebrates caused by whatever pathogen, e.g. fungal or parasite (protozoa), or toxin that the innate and/or the adaptive immune system can fight against, e.g. coccidiosis, toxoplasmosis and infections caused by mycotoxins.
4) Specific viral, parasitic or other infections whose replication (or expansion) the activation of inducible HO-1 enzyme can suppress or even inhibit. These infections cover e.g. malaria, hepatitis C, HIV and Ebola viruses but are not limited to those.
5) All infections whose replication and/or expansion the non-stressful activation of UTPfsAEM can directly or indirectly suppress or inhibit.
6) All infections and related inflammation that the DGA Activation can directly or indirectly alleviate, treat or prevent. These infections cover but are not limited to e.g. ocular infections like conjunctivitis, keratoconjuctivitis sicca, keratitis, lacrimal gland inflammation, gastroenteritis, diarrhea, constipation, diverticulosis, infectious and/or inflammatory bowels diseases (IBD), including but not limited to Crohn's disease and ulcerative colitis.

Specifically, the defense activities that need ATP, ROS and NADPH molecules are enhanced by the DGA Activation. Formation of anti-microbial proteins requires high energy molecules (ATP and NADPH) as well as substrates for anabolic reaction (pyruvate). Additionally, non-stressful increase in the production of carbon monoxide (CO) and biliverdin is protective towards cell and tissue structures in serious infections. CO (and Nitric Oxide (NO) in some species) can be used also in cellular, tissue specific and systemic defenses.

The present invention is useful in replacing excessive use of antibiotics and for use as a non-steroidal anti-inflammatory agent (“by healing”) that can be classified e.g. as a feed additive or a nutraceutical product.

The present invention is useful as an adjuvant by boosting the whole immune system and/or as a combination therapy with other efficient treatments also as a feed additive or a nutraceutical product.

### EMBODIMENTS

In a preferred embodiment of the invention the composition comprises one or more compounds selected from the group consisting of D-glyceric acid, DL-glyceric acid and their salts and esters, as the only active substance or substances.

In another preferred embodiment of the invention the composition consists of one or more compounds selected from the group consisting of D-glyceric acid, DL-glyceric acid and/or their salts and esters, as the sole ingredient or one of the ingredients in a preparation.

A composition comprises one or more compounds selected from the group consisting of D-glyceric acid, DL-glyceric acid and/or their salts and esters for use as an anti-microbial agent or for use as a medicament having an antipathogenic and cytoprotective activity.

A composition comprising one or more compounds selected from the group consisting of D-glyceric acid, DL-glyceric acid and/or salts and esters thereof for use in a method of decreasing infections and related inflammation of humans and animals, including but not limited to live stock (mammals), poultry, and fish. In this use the composition can e.g. improve feed conversion rates in production animal farming.

A composition useful in the present invention may be an oral, topical, parenteral, or inhalable composition for enhancing direct and indirect mitochondrial metabolism comprising one or more compounds selected from the group consisting of D-glyceric acid, DL-glyceric acid and their salts and esters. The composition or compositions for use in the present invention may further comprise a pharmaceutically acceptable excipient. Suitable conventional excipient and/or carriers which can be used in the present invention are known by the skilled person in the art.

The composition may be preparation in the form of a solution, syrup, powder, ointment, capsule, tablet or an inhalable preparation. The composition may be in the form of a solution suitable for parenteral administration.

The various ingredients and the excipient and/or carrier are mixed and formed into the desired form using conventional techniques. The compositions of the present invention may also be formulated with several other compounds. These compounds and substances add to the palatability or sensory perception of the particles (e.g., flavorings and colorings) or improve the nutritional or therapeutic value of the particles (e.g., minerals, vitamins, phytonutrients, antioxidants, antibiotics, NSAIDs, corticosteroids etc.).

The composition for use in the present invention may be a part of a beverage, a food product, a functional food, a dietary supplement, or a nutritive substance.

Said beverage, food product, functional food, dietary supplement, supplementary food, or nutritive substance may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like. Such optional ingredients may be either naturally occurring or concentrated forms.

In an embodiment, the beverage, food product, functional food, dietary supplement, or nutritive substance further comprises vitamins and minerals. In further embodiments, the compositions comprise at least one food flavoring. In other embodiments, the compositions comprise at least one synthetic or natural food coloring.

The composition of the present invention may be in the form of a powder or liquid suitable for adding by the consumer or food producer to a food or beverage. For example, in some embodiments, the dietary supplement can be administered to an individual in the form of a powder, for instance to be used by mixing into a beverage or bottled water, or by stirring into a semi-solid food such as a pudding, topping, spread, yoghurt, sauce, puree, cooked cereal, or salad dressing, for instance, or by otherwise adding to a food, such as functional food.

A packaged pharmaceutical preparation useful in the present invention may comprise at least one therapeutically effective dosage form containing D-glyceric acid, DL-glyceric acid and/or their salt or ester.

An embodiment of the present invention is a pharmaceutical composition comprising an effective amount of one or more compounds selected from the group consisting of D-glyceric acid, DL-glyceric acid and/or salts and esters thereof for use in methods according to present invention.

The present invention is also related to a method of enhancing direct and indirect mitochondrial metabolism in a subject comprising administering an effective amount of one or more compounds selected from the group consisting of D-glyceric acid, DL-glyceric acid and/or their salts and esters to a subject in need. Via decreasing of gastrointestinal tract infections and inflammation the present invention also relates to a method of increasing the muscle yield per gram of nutrition, and preferably simultaneous decreasing of fat content, of humans and animals, and/or alternatively in a method of improving feed conversion rates, i.e. the ratio of nutrition per increase in body weight of animals including but not limited to live stock (mammals), poultry, and fish.

An embodiment of the method comprises administering a pharmaceutical preparation comprising one or more compounds selected from the group consisting of D-glyceric acid, DL-glyceric acid and/or their salts and esters, and a pharmaceutically acceptable excipient. An embodiment of the method comprises administering an oral preparation in the form of a solution, syrup, powder, capsule or tablet.

An embodiment of the method comprises administering one or more compounds via a parenteral solution and topical medicament.

Another embodiment of the method comprises administering one or more compounds via a beverage, a food product, a functional food product, a dietary supplement, or a nutritive substance.

The composition is administered to a subject in need at a dose effective in reducing infection and related inflammation and their resolution. An advantage of the present invention is that the administrable dose is small allowing a convenient dosage to subjects in need. The daily dose in humans may be from 0.1 mg/ kg body weight to 40 mg/kg body weight, such as 0.1, 0.5, 1, 2, 3, 4, 5, 6, 7, 8, 9, 10, 11, 12, 13, 14, 15, 16, 17, 18, 19, 20, . . . , 40 mg/kg, in severe infection or inflammation preferably from 5 to 10 mg/kg body weight once, twice, three or four times a day such as 300 mg-1000 mg per day for 65 kg weighing person. In animals, the daily dosage per kilogram could be also higher per kg or lower e.g. when the animal is bigger than a human.

The present invention is illustrated by the following non-limiting Examples.

### EXAMPLES

The Examples constitute an entirety of findings from various tissues, organs, and whole physiological system from humans and animals in different metabolic states or dosing etc. supporting each other.

In the invention, the UTPfsAEM is non-stressfully activated. This forms the basic solution for a massive number of therapy areas and regimes. The combination of rapidly increased aerobic energy generation and a decline in net ROS generation leads to a decline in infection and a decrease in NF-kB transcription pathway activity (Scheme A). ROS generation is always increased when aerobic energy metabolism is activated. However, in the invention the efficient activation of Nrf2/ARE enzymes enhances ROS scavenging at the same time sufficiently to decrease net ROS generation.

In acute inflammation/stressful attack a rapid increase in ATP production is typically needed for creating efficient inflammatory response (Example 1a-1g). In this kind of a situation temporary increase in net ROS generation and in pro-inflammatory NF-kB activity is inevitable (SCHEME A.). The non-stressful activation of UTPfsAEM works efficiently also in this kind of a situation because it can keep ROS levels at acceptable ranges (Example 2) and simultaneously enhance immune defenses by inter alia increasing energy production capacity of the cells.

As presented in Examples 1-5b the DGA group substances can be used for treating, preventing or alleviating acute infectious diseases and/or disorders via systemic and/or topical treatment. Due fast effect and to novel way of action for anti-inflammation and “immunosuppression by healing the causes” the DGA group substances can be efficiently used also as combination therapies e.g. for strengthening the effects of influenza vaccines, “ . . . therapeutic induction of HO-1 expression may represent a novel adjuvant to enhance influenza vaccine effectiveness.” [1].

In the following it will be shown that the DGA Activation successfully activates, in health, in subclinical stress and in disease, needed mechanisms that are able to enhance the resolution of acute infection and related inflammation. As a proof of concept, in health and in subclinical stress, it is shown that HO-1 enzyme and NADPH production is activated and that inflammation markers and related oxidative stress (ROS) move towards normal levels rapidly. As a proof of concept, in disease/excessive stress state, i.e. in infection, it is shown that inflammation markers are first upregulated to initiate inflammatory response against infection, and that only later ROS and inflammation markers return to normal ranges. Further it is shown that the DGA Activation also leads to resolution of inflammation in preclinical model (Example 1 a and 1g) and in clinical infection (Examples 1b, 1c, 1d, 1e and 1f).

### Example 1a

### Efficacy of the DGA Activation in Intensified Pathogenic Attack Environment—Model in Conjunctivitis, Goblet Cells and Lacrimal Gland Pathology

In this experiment, intensified pathological attacks towards the eye epithelium is used as a disease model for intensified infectious pressure. The protection of the DGA Activation is measured by inflammatory markers. Because the conjunctival epithelium faces outside environment, the conjunctiva was chosen as one important biomarker. In the following this dry eye disease (also known as keratoconjunctivitis sicca) model experiment is called as the intensified pathological attack model or IPA-model.

Epithelial cells typically separate body and its cavities from the outside environment. On top of the eyes epithelial cells line the insides of the lungs, the gastrointestinal tract, the reproductive and urinary tracts, and make up the exocrine and endocrine glands. Pathogens typically attack the body from outside, thus the IPA -model can give indications for wider protection against infectious attacks than just related to the eyes. Goblet cells are another common factor between the epithelium of conjunctiva and e.g. gastrointestinal and respiratory tracts. Goblet cells are found scattered among the epithelial lining of organs such as the intestinal and respiratory tracts. Their function is to secrete gel-forming mucins, the major components of mucus. They are found inside the trachea, bronchi, and larger bronchioles in the respiratory tract, small intestines, the large intestine, and conjunctiva in the upper eyelid. Goblet cells are a source of mucus in tears and secrete different types of mucins onto the ocular surface, especially in the conjunctiva.

Conjunctivitis is inflammation or infection of the surface layer of the conjunctiva. The infection can be bacterial, viral, fungal, and from parasite or caused by some environmental toxin and/or irritant substance.

All animal experiments were carried out according to the Association for Research in Vision and Ophthalmology and the National Institute of Health (NIH) guidelines for the care and use of laboratory animals. The intensified pathological attack model (IPA-model) was induced to naïve seven-to-ten-week-old C57B1/B6J mice obtained from the vivarium at the University of Eastern Finland. In the experiment 4 cohorts of mice were tested, naïve wild-type mice with IPA (n=5), and IPA mice treated topically with either vehicle (n=5) or (topical) DGA (n=5), and additionally IPA treated mice with orally administered DGA with topical vehicle treatment (n=5). IPA was induced by a combination of scopolamine and exposure to a controlled adverse environment (low humidity, high temperature and high airflow for a period of 7 days) in the IPA-mice system. More detailed treatment and administrations were as follows: Group 1: Non-treated IPA mice, Group 2: IPA mice (topical application of PBS to one eye, the contralateral eye served as control), Group 3: IPA mice topical treatment with DGA (aqueous formulation to one eye, contralateral eye served as control), Group 4: IPA mice—systemic treatment with DGA (ad libitum with chow; topical application of PBS to one eye, the contralateral eye served as control).

The DGA Activation was induced be D-glyceric acid (in its calcium salt form). In the systemic administration, the DGA was mixed into chow ad libitum at the concentration of: 1) 65 mg/kg/day for the first 4 days, and at day 5 at the concentration of 130 mg/kg/day prior the dry-eye induction; 2) thereafter the concentration was 130 mg/kg/day for the whole follow-up period of 7 days. In the topical treatment, the DGA was administered topically, as aqueous formulation in PBS. Mice were treated by applying a 5 μL drop 4×daily, for a period of 5 days with the concentration of 15 μg/ml for the first 4 days, and with the concentration of 30 μg/ml for the fifth day prior to exposure to the IPA-mice environment, and for the entire duration of IPA induction in the IPA-mice chamber (7 days).

Tear volume was measured for groups 3 and 4 and later comparison results for group 2 were received. The blood samples were collected from saphenous vein on day 5 (prior the treatment) and on day 7, just prior anesthesia and during the perfusion (cardiac puncture). On day 7, the animals were terminally anesthetized and transcardially perfused. Plasma was separated in all collected blood samples and kept frozen at −80° C. until the measurements. CRP measurements were performed using mouse CRP ELISA kit. The brains, eyes (each eye separately), optic nerves (each nerve separately), lacrimal glands and lids were collected and stored at Experimentica Ltd. The mice were weighed on prior to dry-eye induction and just before the sacrifice. In addition, animals were monitored daily for general health status. The lacrimal glands were embedded into paraffin, sectioned, stained and analyzed for possible pathology.

The combination of scopolamine and exposure of mice to dry environment causes a thickening of the epidermis (FIG. 8/black arrow pointing to the epidermal layer). Thus, any compound/DGA, which lowers the thickening of the conjunctiva has a protective effect. As can be seen from FIG. 7, the DGA Activation possess protective effect both systemically (mixed with the chow) and topically (mixed into eye drops). In both DGA Activation groups the decrease in the thickening of the conjunctiva was some 20 percent compared to relevant control and furthermore the decrease was statistically significant.

Lacrimal gland pathology was evaluated from both eyes of each animal. Three samples (mouse no. 4 right eye (group 1), mouse no. 6 right eye (group 2), mouse no. 18 right eye (group 4)) could not been evaluated due to poor quality of sections. Treatment group 1 (no treated) had the worst pathology found in the lacrimal gland, whereas group 4 had the healthiest lacrimal glands with groups 2 (placebo) and 3 (topical treatment) being in between. FIG. 9 shows the results. Comparison is made to relevant placebo 0-control group. Systemic DGA Activation reduces inflammation statistically significantly in the model (see FIG. 9). In FIG. 10 a tissue sample from lacrimal gland with mild accumulation of inflammatory cells (grade 1) is presented.

In the Example 1 a., also so called goblet cells were measured (see light arrow in FIG. 8). A goblet cell is a glandular, modified simple columnar epithelial cell whose function is to secrete gel-forming mucins, the major components of mucus. Goblet cells are important in the eyes as well as in intestinal tract and similar epidermal tissues. No meaningful differences in the number of goblet cells were detected when the DGA activation groups were compared to 0-controls. Nevertheless, there was a difference between the topical and the systemic DGA Activation groups in the number of goblet cells. This influence is probably related to the positive systemic effects covering both the Local Enforcement and the Double Enforcement from oral DGA administration.

It can be conclude that the DGA Activation in the epidermal/epithelial cell linings of the eyes combined with systemic DGA Activation in relevant organs and in the circulating cells of the immune system managed to keep inflammation lower compared to 0-controls. At the same time decrease in conjunctival thickness shows improved protection against external infectious attacks in the IPA Model. Both the Local Enforcements and Double Enforcement were at play.

### Examples 1b-1g

### Healing from Prolonged and Acute Infections in Respiratory and Gastrointestinal Tracts

In FIG. 13a it has been shown that HO-1 activity increases significantly in healthy subjects (S1 and S4) after 4 days of the DGA Activation in health/subclinical stress. In Example 5b this analysis is extended by measuring the strength of S1 HO-1 expression in peripheral leukocytes (strength=relative gene expression compared to other genes) and it is found that the basal expression of HO-1 in S1 is significant in peripheral leukocytes (=in the Double Enforcement). Further in FIG. 13b it is shown that HO-1 is upregulated also in hepatocytes after the DGA Activation (in pathological cells in vitro/post mortem). From FIG. 13b it can further be seen that both 1.4 μM and 14 μM doses can activate HO-1 gene expression, which is perfectly in line with effective doses for ROS scavenging in FIG. 2b.

In Examples 1 b, 1 c and 1 d it is now shown (by using subject 1 (S1) as an Example), that the DGA activation can lead to alleviation and even resolution of the disease/infection already in 1-3 days. Very likely all the molecular aspects of the DGA Activation are at play in these remarkable alleviation and healing documentations but probably the HO-1 activation facilitated by the help of increased energy metabolism (and following NADH conversion into NADPH) is the biggest explanatory factor.

In Example 1 b, the DGA Activation managed to heal prolonged but relatively mild infection in S1. This very mild disease state was characterized by subdued but persistent fever with varying muscle and other pains during the 2-week period. Towards the end of the period before the DGA Activation muscle pains were slightly increasing. During the disease period patient S1 worked normally but ate 2 or 3 times 1×500 mg of paracetamol during work days to ease general symptoms and fever. Paracetamol gave temporary relief but no cure. Finally, after two weeks of suffering S1 decided to test whether the DGA Activation might help in stopping this prolonged infectious disease state (with unknown origin). As can been seen from Example 1 b prolonged mild disease state disappeared in 2 days.

### Example 1b

### Healing from Prolonged, Mild Infectious Disease

Before initiating any clinical tests full proof of safety was received from 3 week in vivo feeding experiments with rats. This 3-week safety study with 80 rats was conducted in Finnish National Institute of Health with three different D-glyceric acid doses mixed into chow. No adverse health effects were noticed based on normal feed intake and body weight development in all groups and individual rats. Highest doses with rats in the safety study were more than 100 times the doses used for humans in any of the shorter tests presented here. These safety tests demonstrate clearly that the activation of UTPfsAEM is besides non-stressful also clearly non-toxic with used doses.

Efficacy on the DGA Activation on subject 1 (S1) was tested. In 11/2014 S1 got mildly infected by seasonal flu or similar infectious disease. The disease started on Monday after normal weekend. This mild pathological condition didn't seem to go away on its own, i.e. by normal immune response. After almost two weeks of varying pains in different parts of the body (respiratory track, throat, ears, muscles) and very mild fever, muscle pains started to slightly intensify and fever was more clear.

On Sunday (13 days after initiation of the disease) fever was in the morning 36.80 celcius (normal morning temperature (36.2-36.4) and the resting temperature increased to 37.00 celcius during the day (normal temperature at rest and in otherwise stable aphysiological conditions in S1 is 36.60 celcius). Simultaneously muscle pains increased and there was even a slight cramp in gastrocnemius that didn't go away during the Sunday.

Because the immune system of S1 was not able to resolve the infection and because the symptoms were such that the DGA Activation could significantly help muscle pains and simultaneously reduce viral or bacterial pathogenesis, S1 decided to test the possibility that the DGA Activation helps in resolution of the infection.

On Sunday night S1 took two relatively large doses of D-glyceric acid mixed into water to initiate the DGA Activation. (To solute the D-glyceric acid into the water safety tested calcium salt dehydrate formulation was used. Original producer was Sigma Aldrich). Both doses were 3×200 mg. First dose was administrated at 0630 pm and the latter one at 1030 pm, i.e. just before going to bed. The body weight of male volunteer (S1) is 75 kg.

The night after the dosing was relatively like last two weeks during the infection, i.e. “mildly feverish”, but some improvement was possibly observed during the night. In the morning at 0640 am the condition of S1 was much better compared to Sunday morning the previous day. Also, muscle pains were reduced but that had happened also during the two-week disease period and was not necessarily related directly to the DGA Activation. At the same time one must notice that significant difference to the earlier was that fever and muscles pains were markedly more severe on Sunday compared to earlier two-week period and thus it is more likely that the DGA Activation caused marked reduction in disease symptoms.

The dose in Monday morning was reduced to 2×200 mg (after needed relatively large initiation doses).

At 0130 pm fever was at normal level 36.62 celcius (from 37.0 celcius on Sunday). Also, the cramps in gastrocnemius were mostly gone (that were felt in the morning by S1.)

Monday work day (at home office) was normal very busy and intensive day. It continued until 0630 pm. Working was efficient.

At 0700 pm the temperature was normal 36.61 celcius. Physiological condition was normal/good. The cramp in gastrocnemius remained gone. Additionally, during normal night stroll there was no tiredness or sweating like had been the case during last two weeks. The DGA Activation alleviated all symptoms of two-week long infection in 24 hours.

At 1030 pm on Monday night S1 took 3×200 mg of D-glyceric acid calcium salt mixed into water to keep the DGA Activation alive. No fever existed when going to bed at 1045 pm.

Normal wake up at 0640 am on Tuesday. Night went well/normally. No fever during the morning. At 0720 S1 took reduced 2×150 mg of D-glyceric acid calcium salt mixed into water to keep the DGA Activation ongoing. No fever or any other disease symptoms during the work day. At 0345 pm S1 took 200 mg to keep the DGA Activation alive. Weekly double game of tennis at 0700 pm went also well. At 1005 Tuesday night S1 took 200 mg to keep the DGA Activation alive. No fever during Tuesday night or during any consecutive days.

Relatively long infectious disease, containing both viral and bacterial elements, was gone and all the symptoms faded gradually away. Healing process started rather rapidly after initiating the DGA Activation on Sunday. Visible alleviation was seen already in 12-24 hours.

As seen from Example 5a and 5 b, the UTPfsAEM pathway and especially HO-1 gene expression (Example 5b) is strongly but non-stressfully activated in S1 after the DGA Activation. Thus, based on clear prior art evidence on efficacy of especially HO-1 activation in suppressing viral and bacterial infections, it can be concluded that the DGA Activation in minimum enhanced healing from this mild infectious disease.

Obtained very positive result from Example 1 b was not a big surprise because all the symptoms were such that the molecular mechanisms of the DGA Activation might significantly help in curing the infection. But after the positive results from this first clinical in vivo test in disease the door was open for more tests when some flu or similar infection took over S1. As it happened during next 9 months S1 experienced two upper respiratory tract infections.

In Example 1 c the disease prevailed for 3 days before testing the DGA Activation as the cure. In Example 1 d the DGA Activation was initiated only after it started to look obvious that the immune system of S1 could not defeat the infectious disease on its own in reasonable time. In practice, more than one week was waited before initiating DGA Activation in Example 1 d. Additionally, in Example 1 d, the infection was clearly more severe compared to Example 1 c, and possibly was expanding to the lower parts of the respiratory tract. Notably in Example 1 d the disease was not totally defeated as can be concluded from reported mild post symptoms when exposed to cold environment.

### Example 1c

### Healing from Acute Upper Respiratory Infection (Nose and Throat)

After successful healing process in November 2014 (Example 1b), S1 was almost excited when he got acute upper respiratory infection when traveling abroad around New Year 2015. Symptoms: a virus infection in throat and rhinitis had lasted for three days before initiation of the DGA activation. S1 felt also feverish during this three-day period (unfortunately there was no thermometer around during the travel).

Administration on the fourth day of the infection: In the morning at 0940 am S1 took 2×250 mg of D-glyceric acid calcium salt dehydrate mixed into the water. Now, swelling and aching of the throat was quite substantial like had been the case already on the previous night, also some fever was felt.

At 0330 pm S1 took 2×180 mg of D-glyceric acid calcium salt mixed into water to keep the DGA Activation alive. Swelling had reduced a little but this could be due to circadian variation. Third dose of the day (2×180 mg) was received at 0915 pm. Throat continued to feel better towards the night, alleviation of the symptoms compared to the morning and to the previous night was significant.

Next morning at 0715 1×200 mg was received. No fever was observed and general condition was markedly better than on the previous morning. However, some swelling in the throat remained but no pain. On top of the DGA Activation 1×500 mg of paracetamol was received at 0840 am, mimicking combination medication like e.g. Tylenol Cold.

Second 1×200 mg dose was received at 0200 pm. After the lunch at 0100 pm, general condition worsened but that might have been due to relatively active tourist day before lunch. Third 1×200 mg dose of the day was received at 1010 pm. After the relatively busy day general condition was good. (Later it was observed that in infections sufficient therapeutic dose is 2×200 mg several times a day and thus the follow up doses of 1×200 mg might not be sufficient in more serious infections.)

Next morning general condition was almost normal. No administration of DGA group molecules or paracetamol in the morning. In the afternoon, the only dose of the day (1×200 mg) was received at 0200 pm. Feeling was good. Later in the afternoon relatively long walk in the hills. Physical strength was normal and aerobic condition also. No signs of the infection were felt. It was evident that the DGA Activation assisted body's own immune system to conquer the acute upper respiratory infection in 12-48 hours.

As seen from Example 5a and 5 b, the UTPfsAEM pathway and especially HO-1 gene expression (Example 5 b) is strongly and non-stressfully activated in S1 after the DGA Activation. Thus, based on clear prior art evidence (see prior-art references) on the efficacy of especially HO-1 activation in suppressing viral infections, it can be concluded that the DGA Activation in minimum enhanced healing from this acute upper respiratory tract infection.

### Example 1d

### Healing from More Severe Respiratory Tract Infection

Clear symptoms of bacterial and/or virus infection in S1 had started 7 days earlier. Original cause of the disease can also be acquired earlier, i.e. it can have been some hiding pathogen like tuberculosis bacteria that exploits tempting opportunities to attack the body when its defenses are weakened by some physical and/or mental stress (in this case stress could have been also positive stress from overly active days). S1 had had a very nice Summer vacation time in concert and spent the next night after the concert in a crowded train station for 3 hours and then in train for 1.5 hours, i.e. did not sleep at all during Wednesday-Thursday night. On Friday, he was in an activity theme park with the family and next day, on Saturday he drove for 2 hours to a birthday party lasting to Sunday morning hours, and finally on Sunday visited museum that required 500 km of extra driving there and back. Next day, i.e. on Monday morning, the initiation of infectious condition was felt very clearly.

From Monday onwards S1 spent the week until Thursday in the summer cottage and did not rest sufficiently. Thus, the infectious state didn't resolve and probably some new pathogenic attacks managed to test and partially pass the immune system. Only on Thursday evening S1 started full rest because it now seemed obvious that the infection would not resolve otherwise and could even deteriorate further.

On Friday afternoon, fever had risen to 37.40 Celsius that is relatively high fever for S1 whose normal temperature is 36.60 Celsius (C) in rest during the day. Fever remained at 37.40 C until the night. During Saturday, the disease and related fever continued. On Sunday morning, temperature was slightly elevated (36.58 C) compared to the normal morning temperature (36.2-36.4 C). Otherwise the general condition was good on this Sunday morning but during the day fever again rose to 37.15 C by the afternoon and the symptoms in respiratory tract got more severe, resembling intensifying bronchitis or similar inflammatory condition.

By Sunday afternoon it started to seem evident that the immune system of S1 (even assisted by almost perfect rest for 3 days already) would not be able to provide a fast resolution to the potentially serious infectious condition. The Double and the Local Enforcements should be started as soon as possible. (Note: acute fever responses, presented in FIG. 5 and FIG. 11 for S1 have been observed from following administrations of D-glyceric acid in water and calcium salt dehydrate.)

At 12:41 S1 took 2×250 mg of D-glyceric acid calcium salt mixed into water to initiate the DGA Activation. At 0715 pm S1 took 2×180 mg of D-glyceric acid calcium salt mixed into water to keep the DGA Activation alive.

Already before the second dose fever had mostly disappeared and the body temperature of S1 was at 07:10 pm normal 36.60 C. (This reading was verified several times e.g. 36.58 C at 0648 pm and 36.52 C at 0652 pm.) Towards the Sunday night the general condition of S1 improved further. Body temperature at 1015 pm was normal 36.60 C. Last dose of the day 1×200 mg was administered at 1016 pm. Last temperature checkup was done at 1035 pm with reading 36.61 C.

Next morning body temperature was 36.38 C at 0630 am. General feeling in the bronchial tubes was clearly better than on Sunday. At 0658 am 1×200 mg administration started normal intensive Monday work day at the home office due to the infection. During the busy workday the body temperature rose to 36.87 C at highest, which is very normal. Slightly sore throat was also observed during the day.

By Monday night at 0900 pm the body temperature of S1 was normal 36.71 C. Last dose of the day and related fever response was administered at 0908 pm. The dosing was only 1×200 mg and it didn't seem to have any significant immediate impact on fever (FIG. 11). Nevertheless at 1036 pm before going to bed the body temperature of S1 was normal 36.68 C. All in all, the general condition of S1 was healthy.

On Tuesday morning, general healthy feeling continued even though the night sleep was not best possible due to some challenging work issues. Feeling was so healthy that no administration of DGA was needed. Cycling to work went well. During the day, normal healthy condition continued and also towards the night.

By Wednesday it seemed that the 33 hour DGA administration starting on Sunday at 12:41 with higher 500 mg dose and ending on Monday at 0908 pm with 200 mg dose had managed to activate the Double Enforcement and the Local Enforcement sufficiently. With this assistance, the innate and the adaptive immune systems of S1 could get the upper hand against likely multi-pathogenic attack. On Thursday, the normal healthy condition prevailed. However, during Thursday night when sitting long outside on relatively chilly and windy restaurant terrace S1 felt that some of the pathogens tried still to continue their attack, i.e. the body was still at vulnerable state, but importantly healthy condition was restored on its own and no further medication was needed.

As seen from Example 5a and 5b, the UTPfsAEM pathway and especially HO-1 gene expression (Example 5b) is activated strongly and non-stressfully in S1 after the DGA Activation. Thus, based on clear prior art evidence on efficacy of especially HO-1 activation in suppressing viral and bacterial infections, it can be concluded that the DGA Activation in minimum enhanced healing from this more serious infectious disease.

In Example 1b-1d the non-stressful UTPfsAEM activation in respiratory tract epithelial cell linings, relevant systemic tissues/organs and in supporting immune cells, i.e. the Local Enforcement combined with the Double Enforcement managed to resolve infections and related inflammation.

### Example 1e

### Healing from Acute Inflammatory Bowels Disease

In Example 1e the resolution of gastrointestinal tract infection in another subject 4 (S4) has been shown. Like in S1 also in S4 the HO-1 pathway is clearly activated by the DGA Activation. This is indirectly shown by the clear reduction of blood bilirubin in Table 1e. Subject 4 (S4) possesses a history of bowels related inflammations that occur relatively seldom, once or twice a year, but can be very serious leading even to hospitalization for few days. S4 is most of the time healthy and she possesses also good physical condition and keeps healthy diet. During last few years these seldom but acute inflammations in the gastrointestinal tract have been mostly in the colon and caused by inflammation in Colonic diverticula. In these situations, CRP values have typically risen to very high levels suggesting that the symptoms are mostly from serious bacterial infection. The only functioning therapy for inflammatory bowels disease has been the administration of strong intravenous antibiotics.

S4 had already participated in 4-day healthy volunteer test with the DGA Activation. In that earlier non-acute DGA dosing experiment the last dose of D-glyceric acid mixed into water was received previous night, i.e. 10-12 hours before the blood test. In this test the positive effects of the DGA Activation in health/subclinical stress were observed. Especially lowered bilirubin (S-Bil) and bilirubin conjugate (S-Bil-Kj) values compared to 0-control in blood next morning after the DGA Administration were observed. This was the case for all tested subjects including S1 in Examples 1b-1d (see Table 1e below).

TABLE 1e
Changes in Bilirubin values from 0-control in a 4-day healthy human
nutritional test with 5-6 mg/kg/twice a day. DGA calcium salt (DGAcs)
was mixed to 1 dl of water in advance. Healthy subjects with
BMI < 24.9, i.e. normal weight/lean.
Measurements on Monday morning (=0-control) and Friday
morning (=non-acute DGA activation), standard fasting blood test.

| | 4 day changes from zero controls, % | | | |
|---|---|---|---|---|
| | S1 | S2 | S3 | S4 |
| S-Bil | −19.1% | −22.8% | −58.1% | −12.6% |
| S-Bil- | −17.4% | −23.3% | −58.4% | −8.8% |
| Kj | | | | |

Interpretation of lowered bilirubin values in Table 1e: the activation of sAEM last night after receiving D-glycerate mixed into water caused temporary increase in oxidative stress (ROS). Efficient resolution of ROS and related subclinical inflammation was carried out by the activation of HO-1/Nrf2/ARE during the night (SCHEME A). As can be seen from Table 1e this job was carried out excellently. Bilirubin was down for all healthy volunteers because subclinical inflammation was down (and anti-inflammatory HO-1 activity was less needed).

In the following it is now shown that the non-stressful activation of the UTPfsAEM and related HO-1/Nrf2/ARE activation works even in acute bowels inflammation caused by diverticulitis.

Like other tests with unhealthy humans also this test was done because there were no other efficient substances available. When symptoms of increased bowels inflammation/diverticulosis started S4 was on holiday abroad with very difficult access to any specialized doctor not to mention any hospital. Administration of 1000 mg of paracetamol 1-2 times a day helped to relief pains for 2-3 days but the symptoms got worse. Simultaneously with inflammatory symptoms and maybe partially enhancing them S4 experienced constipation.

By Saturday night on the 4^{th} day of gradually increasing acute inflammation in the colon, S4 decided to test whether DGA Activation in combination with paracetamol would assist in resolving this already rather serious inflammation. There were no other available options at that moment. This decision was also supported by confidential information received on positive results with S1 (Examples 1b and 1c).

Saturday night at 0737 pm first 260 mg of D-glyceric acid calcium salt mixed into water was taken with 2×500 mg of paracetamol. Another additional 260 mg dose of DGAcs mixed into water was taken the same night at 1020 pm. Altogether the starting dose was 9 mg/kg i.e. relatively high. High initial dose was chosen because of high level of inflammation.

Sleep during the night between Saturday and Sunday was rather calm and in early Sunday morning there were no serious pains. This was likely due to 2×500 mg of paracetamol taken last night. At 0759 am another 220 mg of DGAcs with water was taken. The inflammation was still there but nevertheless at 0910 am S4 could defecate (almost normally) for the first time in 3 days.

At 0400 pm more serious pain in lower bowels returned. This was likely due to the ending of the effect of pain killers (paracetamol) taken last night. Nevertheless, the pain was clearly less than last night. On Sunday night at 0830 pm, 240 g of DGAcs with water was taken. Still pains in the bowels but they were more manageable than previous night before going to bed. At 0940 pm 2×500 mg of paracetamol was taken.

Monday morning at 0715 am 240 mg of DGAcs with water was taken. Sleep last night was again ok but there was still some pain in bowels left that was felt with movements. During the day the pains eased.

At 0915 pm Monday night 240 mg of DGAcs with water was taken. Clearly less pains compared to the morning. Even without any paracetamol or any other pain killer during the day. No pain killers were needed for the night.

Tuesday morning S4 felt almost totally healthy. 240 mg of DGAcs mixed into water was taken at 0815 am. No paracetamol in the morning. S4 felt healthy during the day also but 500 mg of paracetamol was taken due to neck pain caused by long several hours' car driving during the day. Tuesday night at 1015 pm 240 mg of DGAcs mixed into water was taken. S4 felt totally normal.

Next morning S4 felt totally healthy. All the pains in bowels had disappeared. Last pain symptom was observed during Monday, i.e. more than 36 hours ago. The dose of D-glycerate was reduced to 240 mg once a day. This once a day was continued for next 6 days and for the whole time S4 felt healthy and there were no pains in the bowels.

Immediately after stopping this once a day administration mild bowel pains returned during the 7^{th} day and were very mildly felt still during that night. No paracetamol was needed but twice a day 240 mg of DGAcs for two days resolved the diverticulosis this time. No pains were felt and no DGA Activation was needed after the double doses for last two days. Follow up period without any symptoms and without DGAcs was 7 days, also the weeks thereafter were without any symptoms of bowels inflammation (and no need for DGA Activation).

As mentioned already earlier similar prolonged bowels inflammation had led to hospitalization of S4. Strong antibiotics had to be used for the resolution of this kind of a severe inflammation earlier. This time (because of the compelling circumstances) surprisingly strong anti-inflammatory properties of the DGA Activation had to be used. Some 36 hours were needed to turn the inflammatory process clearly down but some positive symptoms (defecation) were felt already next morning after initiating the DGA Activation on Saturday night.

Non-stressful UTPfsAEM activation in gastrointestinal tract epithelial cell linings, relevant systemic tissues/organs and in supporting immune cells, i.e. the Local Enforcement combined with the Double Enforcement, managed to resolve this acute infection and related inflammation.

### Example 1f

### (Confirmatory Study) Healing from Severe IBD

As explained above subject 4 (S4) possesses a history of bowels related inflammations that occur relatively seldom, once or twice a year, but can be very serious leading even to hospitalization for few days. During the year following acute bowels infection and inflammation described in above (Example 1 e) she was placed by a specialist MD into queue for demanding operation to remove inflammation prone part from her bowels. While waiting for this operation (roughly a year from above described previous serious bowels inflammation (IBD)) S4 got serious IBD again. This happened as a follow up of two-week business trip during which the diet was not at all optimal and working was rather intensive. Symptoms gradually increased during 3-4 days.

By Saturday the symptoms were so severe that S4 could not walk properly. It was decided that she rests for the weekend and goes to a doctor on Monday morning.

Because based on Example 1 e it is already known that the DGA Activation can efficiently reduce IBD in S4, it was decided that S4 starts using DGA immediately to reduce the inflammation. (Also, all other information presented in this application, including all other experiments and dose response analyses were available already, except for Example 1 g.) Initial dose was set relatively high because the inflammation was very severe and because it is known from all other experiments that there were no negative effects for S4 from the DGA Activation. 3×200 mg mixed into 2 dl of water was taken 3 times during Saturday, first dose at 0300 pm, next dose 2 hours thereafter and the last dose 4 hours after the second dose, i.e. at 0900 pm. Inflammatory symptoms remained high but fever reduced from 36.80 Celsius (before DGA activation) to 36.03 by 0939 pm. Paracetamol (1000 mg) was taken after the last fever measurement thus not affecting the decline. Another paracetamol was taken earlier the day at 0346 pm.

Sleep during the night between Saturday and Sunday was rather calm. While in bed in early Sunday morning there were no serious pains. At 0850 am another 3×200 mg of DGAcs with water was taken. The inflammation was still there but nevertheless S4 could defecate (almost normally) for the first time in 2-3 days, again the DGA Activation assisted in restoring this important function of the bowels already in 12-16 hours. (It should be noted that S4 had taken 2-3 times 1000 mg of paracetamol already from Wednesday onwards thus this positive effect was not from paracetamol.)

At 0935 Sunday 1000 mg of paracetamol was taken. By midday Sunday S4 felt rather good in general, but very severe pains in lower bowels were felt in certain movements. At 0115 pm 3×200 mg of DGAcs was taken. Fever was 35.83 at that time i.e. even lower than normal temperature of 36.1 for S4. By 0145 fever was 36.16 i.e. normal. At 0215 1000 mg of paracetamol was taken. Fever remained at slightly above 36 for the whole day. Two doses of DGAcs were taken at 0525 pm and at 0916 pm and 1000 mg of paracetamol at 1005 pm. At night S4 felt better but was not at all healed.

On Monday, the DGAcs administration was kept at elevated level 3×200 mg four times a day. General condition of S4 had improved further, but the inflammation and movement pains were still there. Her CRP level was measured at 0030 pm before going to the doctor. Reading was 85 indicating that the inflammation had been extremely strong during the weekend and that it remained at clearly elevated levels. S4 was prescribed antibiotics (Kefexin/cephalosporin 3×500 mg per day) by the doctor and was ordered for a follow up CRP test at the end of the week on Friday.

S4 bought the antibiotics but did not start the use immediately because she felt better with the elevated DGA Activation. It was decided that S4 goes to an “extra” CRP test on Wednesday, and if her condition keeps improving on Tuesday she does not start use of cephalosporin before this “extra” CRP test.

On Tuesday, the DGAcs dose was significantly reduced to 2×200 mg twice a day because the condition of S4 improved further. There was no fever i.e. the body temperature of S4 varied between 35.92 and 36.20 Celsius during the day. Movement pains were reduced but still existed. Paracetamol (1000 mg) was taken two times during Tuesday.

On Wednesday temperature was normal at 0825 am. At 0830 am 1000 mg of paracetamol and at 1020 am 2×200 mg of DGAcs was taken. Extra CRP measurement was taken at noon (1200 am). CRP was reduced to 22 (from 85 on Monday). This shows that the inflammation was significantly reduced even without antibiotics. Despite recommendations and possibility to start cephalosporin use, S4 decided to continue without antibiotics and with only DGA Activation at least until Friday. (One reason being, that the DGA administration did not cause any side effects, e.g. nausea, like some antibiotics did to her.) More important reason was that the DGA Activation seemed to work even in this rather severe IBD.

On Thursday 2×200 mg twice a day DGAcs administration was continued. On Thursday 1000 mg paracetamol was taken twice. No fever was felt.

On Friday, no paracetamol was taken anymore. DGAcs dose was reduced to 1×200 mg twice a day (morning and before going to bed). Blood CRP was measured at 1100 am. CRP had dropped to 11 showing that clinical level IBD was gone. This is remarkable because in this kind of a situation strong antibiotics have earlier been the only effective medication.

Non-stressful activation of the UTPfsAEM by the DGA administration in gastrointestinal tract epithelial cell linings, relevant systemic tissues/organs, and in supporting immune cells, i.e. the Local Enforcement combined with the Double Enforcement, managed again to resolve acute and this time rather severe IBD.

### Example 1g

### Prevention and Alleviation of Coccidiosis in Broiler Chickens

This 5-week experiment with 351 male broiler chickens was carried out according to official guidelines for the care and use of test animals, and approved by the State Provincial Office of Southern Finland. It was carried out in the premises of the University of Helsinki whose staff also made body weight and feed consumption measurements. Experts from Finnish Food Safety Authority (EVIRA) conducted lesion scoring -analyses and histology. EVIRA also provided counting on the number of oocysts from feces.

In the experiment, there were 4 treatment groups: 0-control, DGA½, DGA1 and Positive Control (P-control). Altogether there were 27 pens and 13 birds in each floor pen. DGA½ group had only 6 pens and other groups (main comparison groups) 7 pens per group. All groups received exactly same feeds and water (ad libitum) except that 0-control did not receive any feed additive or medication. P-control group received coccidiostat medication (Monteban) at recommended doses. In DGA1 group the dose of D-glyceric acid calcium salt (DGAcs) was of similar magnitude as in Example 5a with healthy chickens, i.e. approximately 9 mg/kg/day, and in DGA½ group the dose was half of DGA1 (=appr. 4.5 mg of DGA per kg of body weight per day). Feed additives and medications were given from day 0 until 35 days, i.e. all the time. There were two feeding phases in the experiment: Starter/Grower1 (0-19 days) and Grower1/Grower2 (19-35 days).

Coccidiosis is an infectious parasitic disease of the intestinal tract of animals caused by coccidian protozoa. Besides avian species also mammals can be infected by eimeria species. Species specific eimeria can cause serious health and economic problems in addition to poultry also in cattle and pigs. Coccidiosis symptoms are caused additionally by genus isospora (dogs and cats) and genus toxoplama, e.g. toxoplasma condii that can be harmful also to humans (typically via cats).

Interestingly HO-1 pathway activation as a natural defense mechanism has been reported in coccidial infection caused by eimeria maxima (in vitro) and toxoplasma condii (in vivo) (see [12] and [13]). In reference 13, it is also shown that HO-1 activation suppresses parasite replication. In this Example, it is shown that replication of eimeria oocysts is significantly lower in both DGA Activation groups compared to 0-control (FIG. 16). In Example 5b (FIGS. 13 a and 13 b) it is shown that the DGA Activation can efficiently induce HO-1 expression compared to 0-controls, both on vivo and in vitro. It seems evident, that one mechanism of action of the DGA Activation against protozoal infections is the same as against viral infections [2], i.e. suppression of pathogen replication. This is facilitated especially by cytoprotective HO-1 activation.

Coccidiosis spreads from one animal to another by contact with infected feces or ingestion of infected tissue. Diarrhea, which may become bloody in severe cases, is the primary symptom. Most animals infected with coccidia are asymptomatic, but young or immunocompromised animals may suffer severe symptoms (and even death) that causes serious economic losses in production animal industries. Biggest losses from coccidiosis are typically indirect that arise later from e.g. bacterial and viral follow up infections in animals whose immune defense is first compromised by coccidial infection.

Active pharmaceutical ingredient in Monteban is narasin, an ionophore that is very widely used in professional farming especially in the EU. Narasin is a derivative of antibiotic salinomycin with additional methyl group. Salinomycin is effective against gram+ bacteria but it is also used as anticoccidial agent, especially in the USA where the use of antibiotics as growth promoters is more accepted compared to Europe. According to Norway Food Safety Report 122014, “The EU intended to ban coccidiostats as a feed additive with effect from 2012. Trials were conducted in European countries to identify alternative measures to reduce or prevent coccidiosis in poultry. The conclusion of the EU project was that neither vaccination nor other measures tested could replace the use of coccidiostats in feed.” One aim of this experiment was to find out whether the DGA Activation could be able to provide a sustainable solution for lessening economic loss from coccidiosis in production animal industries. That kind of solution is needed because there is a concern that the use of coccidiostats in feed could result in the development of bacteria with antimicrobial resistance in both humans and animals.

At the age of 15 days all birds were infected with mixture of 3 coccidian protozoa, i.e. eimeria acervulina (100 000 colony forming units (cfu) per bird), eimeria maxima (15 000 cfu per bird) and eimeria tenella (10 000 cfu per bird). The doses were administered orally with 0.6 ml of water to all birds in the experiment. Used eimeria oocysts were provided by Animal and Plant Health Association (APHA/UK). The size of the challenge was optimized to initiate moderate coccidiosis in the birds. Its manifestations should be seen in feed conversion rate and in growth during subsequent 3 weeks left in the experiment until sacrifice of all animals.

As can be seen from the results and in FIGS. 14a, b and c, 15 and 16 the experiment was technically very successful. There were clear and statistically significant deviations in feed conversion rates between 0-control and DGA1 and P-control groups after eimeria challenge. Also, there was deviations in the number of oocysts in treated groups compared to 0-control. Lesion scores were clearly lowest in P-control that is consistent with the fact that this kind of a challenge study is “optimized” for anticoccidial drug that is aimed at killing administered protozoa in the intestines. Furthermore, even though there were observations of bloody diarrhea in some pens 3-7 days after infection, the birds in general managed to recover from the eimeria challenge in all groups that was also one target when determining the size of moderate challenge.

Results in detail: feed conversion rates (FCR) for 14-35 days, 21-35 days and 28-35 days are presented in FIGS. 14a, b and c. FCR is a ratio of feed consumption (per pen) divided by body weight gain in the same pen. Thus, lower FCR rates are better than higher. As can been seen from FIGS. 14a, b and c, FCR is statistically significantly better in DGA1 group compared to 0 -control in all time intervals after the infection (p-values vary between 0.04 to 0.05). These FCR results are also dose dependently consistent, i.e. that DGA½ A group is better compared to 0-control, DGA1 compared to DGA½, and P-control compared to DGA1. Economically most important in respect to FCR are the last two weeks when chickens consume a lot of feed, i.e. periods 21-35 days and 28-35 days. As can be seen from FIGS. 14b and c, the difference between 0-control and DGA1 group widens and simultaneously the difference between P-control and DGA1 group decreases significantly when moving to last two weeks. This positive tendency shows that DGA1 can be an alternative for coccidiostats.

Body weight gain (14-35 days): in both DGA1 group and in P-control group growth was statistically very significantly higher compared to 0-control (respective p-values were 0.002 and 0.0001), (FIG. 15).

Oocysts count: oocysts were measured at 5 different days to have a better understanding of the development during the infection. Feces was collected in the morning for 2 hours from 8 birds in two pens per each group. As presented in FIG. 16, the first measurement was made just prior to infection (day 0), second 5 days after infection, third 7 days after infection, fourth 11 day after and final measurement was 18 days after the infection, i.e. just two days before slaughtering at 35 days. (Results for DGA½ group are not presented in the FIG. 16, because that dose was almost identically efficient in reducing the number of oocysts compared to 0-control as DGA1 group, i.e. lines are overlapping.)

In FIG. 16 there are two important time periods, first one is 5 days from infection (“acute phase”) and the second is 7-11 days (“persistent phase”). In acute phase, as expected, the P-control anti-microbial narasin in Monteban managed to efficiently kill administered eimeria parasites and thus oocyst count rose only very modestly. At 5 days, also in DGA1 (and DGA½) group oocyst count was less than in 0-control, but as can be seen the number of oocysts initially increased clearly more than in P-control. More important, in respect to risk of developing coccidioisis in a flock, is the period after acute phase. As can be seen from FIG. 16 the oocyst count decreased in DGA1 group already in 7 days to similar levels than in P-control and remained there also at 11 days post infection. Notably the gap in oocyst counts between 0-control and DGA1 group widened towards 11-day measurement. (This is in line with increasing difference in FCR ratios between DGA1 and 0-control during week 21-28 days.) Pairwise t-tests, when comparing 0-control against DGA1 and DGA½ groups, showed that the difference in oocyst counts after the infection between 0-control and both DGA groups was statistically significant (p-values: 0.03 for DGA½ and 0.04 for DGA1).

Lesion score: on day 21 (6 days after infection) 81 birds were sacrificed for lesion scoring (and histological samples from 6 birds of interest). Additionally, on day 35, i.e. at the end of the experiment, 12 birds were analyzed (and few histological samples were collected). The lesion scores were counted from three parts of the intestines (upper, middle and caecal counts) and an overall score was counted for each bird. Also, individual weight change during 6-day period after the infection was measure for the 81 birds at 21 days. Results briefly: as expected individual weight change between 15-21 days correlated negatively with overall lesion score. In 0-control group overall lesion score was highest, follow by DGA1 group, DGA½ group and P-control. The difference in overall lesion score compared to 0-control was statistically significant only in P-control. At 35 days, no lesions from eimeria were observable anymore. This observation was as expected and it is in line with oocysts counts, i.e. that in all groups the number of oocysts were almost zero at 33 days (FIG. 16). One can conclude that coccidiosis was over in all groups by the last week of the experiment.

Mortality after 14 days: in DGA1 group no mortality, in 0-control 2 birds died (days 15 and 35), in P-control one bird died at 21 days, and finally in DGA½ group no mortality but one birds was clearly ill at slaughtering. In earlier broiler chicken experiment without infection (see Example 5 a) the situation was similar in respect to late mortality: no mortality in DGA groups and 1-2% mortality in other groups.

It can be concluded inter alia that the DGA Activation, especially with similar dosing than in DGA1 group, is very potential candidate when looking for alternatives in replacing coccidiostats in commercial broiler chicken farming. Very positive for the future use of the DGA Activation against coccidiosis was that in both DGA1 and DGA½ groups the number of oocyst declined to almost similar levels than in P-control in 7 and 11 day measurements, and that simultaneously in 0-control the number of oocysts remained at elevated levels. Shown depression of protozoa replication supports the idea that the DGA activation can depress also virus replication e.g. by activating HO-1 pathway.

Based on oocysts count (33 days) and lesion scoring at 35 days, one can conclude that coccidiosis was over in all groups by the last week of the experiment. Nevertheless, FCR was clearly better and even improving in DGA1 group compared to 0-control during the last week (FIG. 14 c). This is a clear sign that the DGA1 group recovered from typical follow up infections of (induced) coccidiosis much faster than the 0-control group. This observation is nicely in line with Examples 1a-1f, where it has been shown that the DGA Activation is efficient in supporting immunological defenses against pathological attacks on epithelium in the eyes, in respiratory tract and in intestines. (In P-control there was no real coccidiosis and thus better FCR during 28-35 days in FIG. 14 c does not represent any real recovery.)

Commercial broiler chicken farming lasts on average slightly longer than the 35 days, e.g. in Finland until 37 days. In these slightly longer periods improving relative FCR for DGA1 group towards the end of the test period can result even to better efficacy outcome for the DGA Activation under coccidial pressure. Also, decline in late mortality improves FCR.

Based on Examples 1e, 1f and 1g combined with other presented evidence on the enhancement of the inflammatory response (Example 2/acute ROS up regulation, Example 3/CRP in disease up, and Example 5b) and the anti-inflammatory effects (Example 1 a-d, Example 2/long term ROS downregulation, Example 3/CRP in health down, Example 4 and Examples 5a and 5b) it can be concluded that the DGA Activation can activate cellular and tissues specific defenses and also systemic immune defenses and their control against bacterial, virus, fungal or parasite (protozoa) infections in gastrointestinal tract (GI). These healing effects in GI are so strong that likely the non-stressful activation of UTPfsAEM and related enhancement in GI nervous system enhances also the motility and thereby promotes healthy gut microbiota [14], which can compete against pathogens and indirectly support the immune systems.

Especially the HO-1 downstream metabolites and pathway is activated (SCHEME C, FIGS. 13a and 13b). Epithelial defenses in respiratory tract (1b-1d), gastrointestinal tract (1e-1g) and in ocular epithelium (Example 1a) are enhanced significantly. All in all, it can be concluded that the DGA Activation can alleviate and even heal wide range of viral, bacterial, protozoal and other infectious diseases. Both the Local Enforcement and the Double Enforcement are at play as can be seen from Examples below and above. Main intracellular pathway and molecular mechanisms underlying this remarkable healing effect are presented in SCHEME B and SCHEME C.

### Example 2

### ROS Generation and Increase in Energy Metabolism in Optic Nerve Astrocytes

“Evaluation of efficacy of the DGA Activation on cell viability in response to oxidative stress in rat primary optic nerve astrocytes and the production of reactive oxygen species (ROS)”. Excessive ROS generation is linked to acute inflammation e.g. via NF-kB and prostaglandins. (Prostaglandins are hormone-like auto- and paracrine mediators of multiple important activities in cells and tissues.) Excessive amount of ROS activates NF-kB via cascade of invents and increases prostaglandins and the production of some cytokines. NF-kB activation, prostaglandin production and their coordinated action with cytokines form an important common link between oxidative stress (Example 2), CRP/inflammation (Example 3), observations with fever (Example 4), and finally observations with stress hormones cortisol and corticosterone, i.e. glucocorticoids, release (Example 5a).

Additionally, excessive ROS formation can also cause NAD+ depletion. This is because NAD+ also serves as a substrate for both the sirtuin family of NAD-dependent histone deacetylases and the DNA repair enzyme, PARP (poly(ADPribose) polymerase). Less ROS can thus in some situations also help fighting acute infections and inflammation by sustaining NAD+ -pool higher and thus facilitating cytosolic ATP production.

For proving the efficacy of DGA Activation in reducing ROS in infectious disorders an in vitro study using rat primary optic nerve astrocytes has been conducted. In this study the objective was to assess the efficacy of D-glyceric acid in scavenging the acute production of reactive oxygen species (ROS) (15 min-240 min) in response to 1) normal metabolism (change of the nutrition/media) and additionally in response to 2) extra oxidative stress/disease caused by bolus administration of tBHP (tert-Butyl hydroperoxide) in primary optic nerve astrocytes. As an important part of the study the cell viability was estimated 6 hours after tBHP administration by two methods, i.e. LDH and MTT assays. Cell viability was measured using the in-house MTT assay (of Experimentica Ltd., Finland) and CytoTox96 non-radioactive cytotoxicity assay (G1780, Promega) in response to oxidative stress primary optic nerve astrocytes. The production of reactive oxygen species (ROS) was measured using CellROX Green (Invitrogen) and isolate RNA in RNALater solution (Ambion).

Primary optical nerve astrocytes where obtained from 4-week-old male Wister rats (6 animals' altogether) and where cultured to obtain sufficient amount of cells for the experiment.

Prior study preparations to find suitable tBHP concentration: The astrocytes where seeded at 5000 cells per well and allowed to grow for 48 hrs to mimic the study. On day 2 the medium was removed and the cells were exposed to a dose response range of tBHP in fresh medium for 6 hrs. The MTT and LDH assay were performed to assess cell survival using the Victor microplate reader. The raw data was given to patent applicant for assessment. A concentration of 85 μM tBHP was chosen to mimic extra stress/disease model that caused suitable roughly 20-25% cell loss compared to 0 tBHP.

In the experiment cells where plated at 5000 cells per well and allowed to adhere for 24 hrs prior to the administration of compounds for 48 hrs (replenished every 24 hrs). After 24 hrs the compounds were removed, and readded with new medium for 1 hr, prior to the administration of 85 μM tBHP (FIG. 3c). MTT and LDH assay were performed after 6 hrs and ROS detection using CellROX green was performed over a time course of 4 hrs.

D-glyceric acid was mixed into the medium in beforehand in soluble calcium salt format (DGAcs). After mixing the calcium is liberated to the medium from the D-glyceric acid. D-glycerate is formed. (The calcium concentration of the medium was more than 100 hundred times higher compared to calcium liberated from administered D-glyceric acid and thus could not have any significant effect on these results.)

As can be seen from FIG. 3a, observed ROS generation in 0 tBHP groups, i.e. in healthy conditions with moderate metabolic stress, is lower in 14 μM of D-glyceric acid compared to 0-controls. (0-controls were administered medium only.) From FIG. 3c it can be observed that the administration of the DGA was initiated already 48 hours before. Thus, observed ROS is partially a cumulative count (partially because the half-life of ROS is relatively short, and thus most of the ROS generated during 48 hours had been already neutralized before the measurement in both groups.)

As can be further seen from FIG. 3a, the situation in respect to ROS seems to be similar in the disease model i.e. in extra oxidative stress induced with (mildly toxic) 85 μM tBHP administration. ROS level after 15, 30 and 60 minutes after the bolus administration of 85 μM tBHP are statistically significantly lower in DGA groups compared to control groups without the DGA Activation. It can be concluded that the baseline result of reduced oxidative stress by DGA Activation holds nicely also in disease/extra stress.

So far, the results of this Example 2 have shown that the DGA Activation can efficiently reduce ROS generation and/or keep it lower in optic nerve astrocytes in both health and disease model in vitro. Thus, in health and in subclinical stress the DGA Activation can reduce excessive ROS formation and as a result also reduce inflammation and stress reaction mediated by NF-kB, prostaglandins and cytokines.

As can be seen from FIG. 3a, the study with rat primary optic nerve astrocytes confirmed the ability of D-glycerate group to reduce ROS generation statistically significantly compared to relevant controls in health/in subclinical stress, i.e. without tBHP administration but with normal metabolic stress. This effect was similar than seen earlier with hepatocytes in humans (FIGS. 2a and 2b) and used dose (14 μM) was the same as in hepatocytes. All in all, it can be concluded that antioxidant scavenging (related to AREs) is activated by DGA also in rat primary astrocytes. Furthermore, based on gene expression findings in FIGS. 13a and 13b this is beyond doubt due to the non-stressful activation of UTPfsAEM.

Here it is further and importantly shown that the acute ROS creation in astrocytes is increased by the DGA Activation in extra stress situation caused by 85 μM tBHP. Mathematically expressed, in D-glycerate (DGA Activation) groups the average ROS generation increases in 15 minutes (from 0 min to 15 min) approximately 30% in tBHP administration group compared to without tBHP, and only approximately 20% in similar control group comparison. (This surprising result of acute increase in ROS can be achieved by assuming perfectly realistically that before the bolus administration of tBHP (at time=0 min) the average ROS level in the 0-control groups (2×6 repeats) and in DGA groups (2×6 repeats) respectively was on average the same.)

Because aerobic energy metabolism produces roughly 90% of ROS, observed increase in relative ROS generation must be due to increased aerobic energy metabolism due to the DGA Activation (see “phases” in SCHEME A). This conclusion is also in line with other findings in enhancement of aerobic energy metabolism by the DGA Activation. This entirety is also in line with shown fast but moderate in vivo increase in energy metabolism by the DGA Activation like shown by TSH decline (FIG. 1). Higher energy production without increase in long term net ROS can protect astrocytes in multiple ways. Notably this increase in energy metabolism seems to give protection to the cells both in health and in disease state because the cell viability increases in both compared to the 0-controls (FIG. 3b).

All in all, in disease/extra stress situation acute ROS generation is increased by the DGA Activation and at the same time longer term excessive ROS generation is kept under control. When it is considered that in this study the cell viability after tBHP administration was clearly higher in the DGA Activation group, this temporary increase in ROS seems to protect astrocytes in this model. (Similar difference prevails also in 30 and 60 minutes, but in longer term comparison different cell viabilities might disturb the ceteris paribus assumption in the comparison.)

Acute protection from induced stress by increasing energy production and temporary increased ROS generation extends the therapeutic use of DGA Activation towards acute diseases and related inflammation, e.g. viral keratitis most often caused by herpes simplex virus.

### Example 3

### Effect on CRP in Health and in Disease/IL-6 Third Proof of Concept of the DGA Activation: CRP in Health and in Disease Experiments

C-reactive protein (CRP) is so called acute phase protein related to inflammation. Its production in the liver is activated by acute infection, inflammation and/or trauma. Main activator of CRP production in the liver is IL-6 (interleukin-6). In disease state, e.g. after pathogenic attack or after some trauma, pro-inflammatory CRP level rises rapidly and can reach very high levels compared to healthy conditions. The half-life of CRP protein is 18 hours, and thus the level declines relatively rapidly after the resolution of the disorder. But if the disease state is intact, new IL-6 is released by macrophages and other cells of the immune system and CRP level can remain high for longer time.

All in all, blood CRP -level is a sensitive indicator of very wide range of inflammatory processes and responses and thus reliable as a general indicator. The challenge with CRP is that it is unspecific and that it can be a pro-inflammatory and an anti-inflammatory marker. In general, it can be said that pro-inflammatory NF-kB transcription pathway is typically activated when CRP levels are high and it probably is down-regulated when CRP levels are low or when they decline substantially.

In healthy conditions, i.e. in subclinical stress, the DGA Activation should optimally reduce blood CRP -levels because DGA Activation reduces stress locally and via systemic effects, and thus can tackle subclinical inflammation and infections. (Mechanistically IL-6 release by macrophages and other cells is reduced leading to decline in CRP.) On top of this general tendency, there is also a further possibility, in healthy condition, that activation of anti-oxidant and anti- and pro-inflammation pathways by the DGA Activation could trigger slight CRP rise in “low CRP subjects” and even increase very low CRP -levels.

In this Example, CRP results from 3 separate preclinical tests are presented and analyzed. First 4-day CRP test was conducted with 8 healthy mice using individuals as their own controls. Second 7-day CRP test was conducted in discontinuous disease model. And third also 7-day CRP test was conducted in continuous disease model. CRP present in mouse serum samples were analyzed using mouse CRP ELISA kit (GWB-3B960E, Genway Biotech Inc.). All blood samples were collected at the same time of the day.

For evaluating possible effect of the DGA Activation in health, a 4-day test with healthy mice in Charles River Labs, Kuopio test facilities were conducted. In this test (prior initiation of the MPTP -model) the DGA was mixed into chow at the concentration of 65 mg/kg/day.

The CRP results in health are presented in FIG. 4a. As it can be seen from the FIG. 4 a, after 4-day treatment/DGA Activation CRP level in each mouse was typically lower compared to starting level. (Starting CRP -level for individual mouse is shown as a black bar and the ending CRP-level in grey.) In pairwise one-sided t-test this overall decline in CRP was statistically significant (p-value=0.04). Thus, it can be concluded that in healthy conditions, i.e. in subclinical stress, the DGA Activation reduces blood CRP -levels in general. This is because the DGA Activation reduces stress locally and via systemic effects, and thus can tackle subclinical inflammation and infections (SCHEME A). (Additionally, on top of this general effect of reduction in subclinical CRP values possibly “too low” CRP values rose, because in FIG. 4 a there exists a clear tendency towards the average 0-control/starting CRP -level (=0.222, solid line in the graph) after the DGA Activation. All CRPs above the average (5 out of eight) decline towards the average CRP and two of the thee CRPs below move up towards the CRP average.)

In two other CRP tests, the effect of the DGA Activation in two disease models, continuous vs. discontinuous was analyzed. In FIG. 4b, results from discontinuous MPTP/PD disease model, and in FIG. 4c, results from continuous IPA/Dry Eye disease model. Logarithmic change of blood CRP during the 7-day disease follow up period of each mouse in all groups was calculated and the averages are presented in FIGS. 4b and 4c. (In both disease models, there was a 4-day priming with 50% DGA dose before activating the disease model and CRP test.)

As can be seen from FIG. 4b, in discontinuous disease model CRP declined statistically significantly in the DGA Activation compared to 0-control with no DGA Activation (p-value=0.015). This indicates healing from PD/MPTP disease model. Notably in this discontinuous disease model the CRP decline was statistically even more significant when the CRP change in individual mice was compared (pairwise test, p-value=0005). This discontinuous disease model resembles the challenge in Example 1 g.

On the other hand, FIG. 4c shows clearly that in continuous disease model the DGA Activation can enhance inflammatory response. In this IPA model CRP increased statistically significantly in the DGA Activation compared to 0-control with no DGA Activation (p-value=0.027). In simplified terms cytokines (especially IL-6) continue to be elevated causing the liver to keep producing CRP more proteins compared to 0-controls.

Brief description of the disease models: 1) continuous IPA -model (see Example 1a) and 2) discontinuous MPTP/PD -model. MPTP (1-methyl-4-phenyl-1,2,3,6-tetrahydropyridine) is a neurotoxin precursor to MPP+, which causes symptoms of Parkinson's disease (PD). In the MPTP -experiment there were two test groups, 0-control and (systemic) DGA Activation group, and as shown in [15] serum CRP should return to normal in 0-control in 5 days. In both experiments blood samples for CRP measurements were taken from the mice immediately prior to the disease induction and at the end of the 7 day disease model. In the MPTP -experiment there were two test groups, 0-control and systemic DGA Activation group (8 mice in both groups). In both disease models the systemic administration of the DGA was identical, i.e. mixed with the chow like in Example 1a. and 1g. (Chow and water available ad libitum.) As in all experiments, also in MPTP -model animal experiments were carried out according to the National Institute of Health (NIH) guidelines for the care and use of laboratory animals, and approved by the State Provincial Office of Southern Finland. Altogether 16 male eight to twelve-week-old, C57B1/6J mice, purchased from CRL Germany, are used for the experiment. Animals are housed at a standard temperature (22±1° C.) and in a light-controlled environment (lights on from 7 am to 9 pm) with ad libitum access to food and water. Diet consumption per cage is monitored carefully before and during the study follow-up period. Average consumption of the diet with or without DGA is used to calculate the needed concentration in the diet to achieve desired exposure levels. MPTP hydrochloride (Toronto Research Chemicals) is given twice a day at the dose of 15 mg/kg in saline i.p.

at 3-h intervals on two consecutive days (Days 0 and 1), the total amount being then 60 mg/kg. The number of mice per MPTP treated group is 8 (n=8).

In both disease models the 4+7-day DGA Activation improved survival and health parameters but notably as can be seen from FIGS. 4b and 4c the change in mice CRP levels deviated significantly. In continuous disease induction the CRP levels were on average higher (compared to starting level) in the DGA Activation group compared to 0-controls at the end of the test, and in discontinuous model wise versa. These differing differences were even statistically significant in both models.

Elevated CRP levels compared to control groups in continuous disease model is perfectly in line with observed increase in acute ROS generation in disease state in Example 2. This finding gives clear support to the claim that in (continued) pathological attack the DGA Activation can keep the immune system, i.e. the Double Enforcement more activated compared to control. Furthermore, it seems to lead with other positive effects (“Local Enforcement”) into subsequent beneficial therapeutic results. Conversely in health and/or subclinical stress the Double Enforcement leads to reduced CRP values and in practice immediate health effects by reducing stress and inflammation level of the body, i.e. in healthy state “phases” in SCHEME A materialize almost instantaneously and resolution of subclinical inflammation/stress is immediate.

Blood IL-6 level has been measured only in healthy subjects thus far and in combination with glucocorticoids. For IL-6 results see the description of FIG. 6b and Example 5a. On top of being a CRP activator IL-6 possesses also some feedback/stabilizing anti-inflammatory functions. IL-6's role as an anti-inflammatory cytokine is mediated through its inhibitory effects on TNF-alpha (TNFa) and IL-1, and activation of IL-1ra and IL-10. On the other hand, IL-1 and TNFa can in certain conditions increase IL-6 secretion. As already explained earlier TNFa and IL-1 are important activators of NF-KB transcription pathway. In this complicated self-balancing system, it is not possible to make definite conclusion based on only IL-6 levels. Except, that in stable (inflammatory) state a reduction in IL-6 level is an indication of reduced stress/inflammation.

### Example 4

### Acute Reduction of Fever Fourth in Vivo Proof of Concept of the DGA Activation: Thermoregulation/Fever

Fever is directly related to infections and related inflammation through the immune system. If the DGA Activation can efficiently reduce fever, it is a further proof of concept for anti-infectious and anti-inflammatory effects.

Body temperature in vertebrates is controlled by hypothalamus. Typically so called pyrogens cause a release of prostaglandin E2 (PGE2) that acts on hypothalamus to increase temperature set-point. Pyrogens can be exogenous e.g. bacteria derived lipopolysaccharides (LPS) or then endogenous cytokines. Major endogenous pyrogens are IL-6 and IL-1 (alfa and beta). Minor pyrogens include e.g. some interferons, TNFbeta and Macrophage inflammatory proteins. TNFa is an indirect pyrogen that acts through IL-1. Typically, also exogenous pyrogens act through endogenous pyrogens released by the cells of the immune system.

“Fever generator” PGE2 is generated by PGE synthase enzyme in an isomerase reaction from common precursor PGH2. PGH2 arises from the arachidonic acid. This pathway (as it relates to fever) is mediated inter alia by cyclooxygenase-1 and -2 (COX-1 and -2) enzymes. COX-2 is the inducible form of the enzyme that is reactive to stimuli e.g. infection and/or excessive ROS formation. COX-2 is also regulated by NF-kB transcription pathway.

Like is typically the case in paracrine and autocrine regulation and in endocrine related regulation also in thermoregulation numerous negative and positive feedback and control mechanisms are in place. IL-6 has been identified as an important positive modulator of PGE2 effect on hypothalamus. All in all, the regulation of temperature (set point) is immune-system based and contains both local (PGE2) and systemic (IL-6) elements. Thus, changes in e.g. bacterial, viral, protozoal and other exogenous infections, and endogenous oxidative stress and inflammation can induce fever by increase in cytokine release.

In Example 4 acute 15 min, 30 min, etc. fever responses of the DGA Activation in two individual under viral and/or bacterial infection (subjects S1 and S4 from e.g. Examples 1b-1f) were measured. Results are presented in graphical format in FIG. 5 and in numeric format in FIG. 11. The measurements of body temperatures after the DGA Activation were made in physiological state where infections (diseases) continue but they have nevertheless more or less stabilized. In this kind of a situation the immune system is activated above healthy and/or subclinical level. The objective was to verify possible spillover effects from reduction in inflammation and in oxidative stress towards fever. At the same the target was to gain more info on the size of suitable administration. (Before initiating any human tests full proof of safety was received from 3 week in vivo experiments with rats. The doses used in these clinical tests are at maximum less than 5% of the safe doses with rats in the 3-week safety tests.)

The results in Example 4 are an additional remarkable proof on the fast impact time of the DGA Activation. Fever reduces already in 15-30 minutes and notably returns almost to the starting level in 1 hour or at least in 1.5 hours. Decline of fever is modest but it is nevertheless statistically very significant.

In a disease state the return of the fever to the starting level is an indication that the fight against infections continues, mechanistically immune cells continue to produce endogenous pyrogens (e.g. IL-6 that induced CRP production in previous Example 3, see also FIG. 4 c) and possibly also that the (continued) infection continues to cause excessive ROS generation in infected tissues that induce PGE2 production through COX-2 activation induced also by NF-kB. Pro-inflammatory fight continues that should eventually lead to resolution of the infection.

In stabilized disease the DGA Activation leads to temporary reduction of fever (an indication of successful enhanced fight against inflammation) that leads eventually to the resolution of the inflammation and cure from the disease. In terms of “phases” presented in SCHEME A, the resolution of the disease in clinical illness lasts clearly longer than in subclinical stress and it typically needs several rounds of DGA Activation. In severe cases one to two weeks of DGA Activation several times a day and in combination with other effective medications might be needed.

Because the effect on fever is temporary and subdued and because the effects of prostaglandins are only local, it is postulated that the reduction in fever doesn't necessarily come from the reduction on PGE2 production, but likely from temporary reduction in IL-6 release, i.e. fever reduction is probably more related to the systemic Double Enforcement than to Local Enforcement. Interestingly this mechanism differs from many NSAID medications that reduce fever through inhibition of COX-2 enzyme, and subsequently the amount of PGE2.

It is important to realize that the DGA Activation reduces fever because it reduces the cause of fever, not by inhibiting generation of fever like many other immunosuppressive agents typically do.

### Example 5a

### Thyroid and Glucocorticoids In Vivo Endocrinologic Proof of Concept of the DGA Activation:Glucocorticoids, TSH and T4 in healthy volunteers

The stress hormone cortisol (and/or corticosterone) is segregated by the adrenal cortex within adrenal glands. Its segregation is regulated by complicated hormonal cascade starting from hypothalamus (corticotrophin releasing hormone, CRH) via anterior pituitary gland (ACTH) towards adrenal cortex. This system includes also e.g. negative feedback loops from released cortisol in the circulation.

IL-1 has been identified as the major cytokine that causes activation of the production of this endocrine hormone synergistically with CRH through ACTH. Glucocorticoid suppresses the immune system and inflammation through e.g. inhibition of NF-kB and it aids in metabolism of fats, proteins and carbohydrates, used in energy metabolism. Glucocorticoid is mainly released in response to stress and low blood-glucose concentration. On top of suppressing inflammation glucocorticoid can also modulate the inflammatory response in certain specific situations.

Persistent too high glucocorticoid blood levels are an indication of inflammation. Also, psychological stress can keep glucocorticoid levels elevated. Correct endogenous glucocorticoid supply and its concentration in blood are important in maintaining normal physiological situations as well as in pathological and in stress conditions. Thus, also too little glucocorticoid can be very harmful.

For our purposes the main effects of glucocorticoids, i.e. 1) suppression of inflammation and 2) enhancement of energy metabolism of fats, proteins and carbohydrates, are very suitable and in fact almost perfect biomarkers of biological effects in health/subclinical stress. Both biomarkers should reduce the need for glucocorticoid segregation by adrenal glands after the DGA Activation in healthy humans and in broiler chickens. This is especially evident in the case where subject's cortisol level is in the upper part of the normal fluctuation range. (See the “PHASES” and Examples in SCHEME A for further explanation why cortisol and glucocorticoid levels should decline in healthy condition.)

The objective of the study was to find out, if stimulation by hormones explained part of the relatively strong effects of the DGA Activation in health/subclinical stress. Additional target of this study was to find out how fast the effect in hormonal response is in vivo.

Because thyroid gland and thyroid hormones are important and direct regulators of energy metabolism, it is important to measure also TSH and T4 when measuring and interpreting the effect of the DGA Activation on metabolically more complicated cortisol. As already noticed above the decline in TSH (FIG. 1) is a clear proof of concept of more sustainably functioning energy metabolism.

First, two healthy human volunteers (subjects S1 and S9) were tested. (Before initiating any human tests full proof of safety was received from 3 week in vivo experiments with rats. The doses used in these clinical tests are at maximum less than 5% of the safe doses with rats in the 3-week safety tests.)

Blood levels of cortisol, thyroid stimulating hormone (TSH) and thyroxin, i.e. thyroid hormone T4 (T4) from standard EDTA blood sample were measured. 0-control and 2 day follow up measurement were conducted at approximately 08.55 am and the same day, 1.5 hour follow up, at 10:25 am. All measurements were conducted in “half fasting” state, i.e. half of the normal breakfast eaten 1.5 hours before the collection of the blood sample, and the other “half” immediately after 0-control measurement. First measurement without the DGA Activation served as the 0-control for both follow up measurements with the DGA Activation. First “DGA measurement” was conducted after 1.5 hours from 0-control (“fast effect”) and the second after 2 days (“persistent effect”). All measurements were made in as comparable situations as possible (ceteris paribus). Last dose was 300 mg of D-glyceric acid mixed into water (or 430 mg of D-glyceric acid calcium salt dehydrate mixed into water), and otherwise the daily dose was 2*225 mg D-glyceric acid calcium salt dehydrate mixed into water. Male volunteers S1 and S9 weigh 75 kg and 80 kg respectfully.

Results on blood TSH can be found in FIG. 1 and the result on blood cortisol in FIG. 6a. From the results related to TSH one can see that the administration of DGA clearly has a positive impact on energy metabolism of healthy human. Furthermore, this effect is fast. Cortisol measurements support the observation that the DGA Activation enhances energy metabolism. Furthermore, they also support the claim that this activation is non-stressful, i.e. does not cause a (permanent) increase in ROS. Clear 20-40% percent level decrease in blood cortisol also evidently indicates a reduction in subclinical inflammation is both S1 and S9.

In a confirmatory study, healthy broiler chickens are studied. FIG. 6b confirms glucocorticoids reduction in vertebrates in general by showing that in broiler chickens (N=30) blood corticosterone (=same as cortisol in humans) declines by some 40% after 3 week DGA activation compared to 0-Control. This decline in corticosterone is statistically significant (p-value =0.021) and was observed also in blood at 28 days. Also, lower average IL-6 levels were observed in broiler chickens after 21-28 days of DGA Activation compared to 0-control. This average reduction was clear but not statistically significant (one-sided test p-value=0.12). In health/subclinical inflammation reduced IL-6 levels mean less infections and related inflammation. The challenge with IL-6 and other cytokine levels is that they are not sensitive indicators of infections or inflammation in subclinical conditions. (Feeding tests with broiler chickens (altogether 144 birds) were conducted in Natural Resource Institute Finland.)

From the results of Example 5a, it can be seen that in humans the blood cortisol levels decline already in 1.5 hours after the DGA Activation. This fast effect is statistically significant and more than 22% for both S1 and S9. Furthermore, the expected effect of the DGA Activation remains mostly the same after 2 days in S1 and S9. Simultaneously the level of T4 remained stable (average change from 0-control was less than 1%) and notably the level of stimulator hormone TSH declined by −24% (fast effect) and −13% (after 2 days). The decline in TSH is also statistically significant.

Furthermore, in broiler chickens the effect of the DGA Activation to reduce stress and enhance metabolism was seen even after 21 days by statistically significantly lowered glucocorticoid levels (see FIG. 6b). As already noted above, lower corticosterone was observed also in blood at 28 days. (This latter result was also statistically significant when so called “%-bound” results were tested against 0-control.)

Despite complicated interrelationships and challenges in forming exact test hypotheses in relation to lower glucocorticoid levels, it can be firstly concluded that the systemic need for hormonal suppression of the elevated activity of the immune system due to infection/inflammation is clearly down after the DGA Activation (“phases” in SCHEME A). This is seen by the decline in cortisol in humans and corticosteroid in broiler chickens in all measurements in health under the DGA Activation. In other words, these results are a clear proof that the DGA Activation can reduce subclinical inflammation fast and that this effect seems to last at least for several days and even for several weeks. This conclusion is directly and strongly supported by independent Examples 1 (ROS decline), 2 (CRP decline) and 3 (reduction in fever), and additionally by earlier ROS studies conducted in hepatocytes, see FIGS. 2a and 2b.

Secondly, it can be concluded that the decline in cortisol and the TSH decline combined with stable T4 are almost perfectly in line with our claim that the Local Enforcement works efficiently in the enhancement of intracellular energy metabolism without excessive ROS and oxidative stress. Local immune defenses are enhanced which reduces the need for systemic immune response. This entirety importantly liberates systemic resources for other acute immunological tasks.

Furthermore, the observed reduction in glucocorticoids combined with above and other similar evidence is a clear proof of concept that the DGA Activation can efficiently reduce subclinical inflammation. Both the Local Enforcement and the Double Enforcement are active.

There is additionally a complicated stress (glucocorticoid)/NADPH dependent relation between glucocorticoid and the conversion of T4 into much more active form T3. Without going into details, the net conversion of T4 into T3 is reduced in stress, and this reduction is related to depletion of anti-oxidant NADPH pools in the liver and other tissues where this conversion mainly takes place. Thus, elevated level of glucocorticoid (when due to oxidative stress) likely reduces T4 conversion into T3. This may decrease the positive effect of thyroid hormones to energy metabolism. Because the DGA Activation enhances NADPH formation and reduces oxidative stress (in the liver), it can be concluded and predicted that, if anything, the DGA Activation increases T4 conversion into T3. This gives further support to the claim that sAEM is activated at cellular level.

### Example 5b

### Genome Wide Rank and Relative Strength of Identified Genes

In the FIG. 13a it is shown that UTPfsAEM gene expressions are activated markedly in peripheral leukocytes after 4.5 day DGA Activation compared to 0-control. The analysis of selected genes was conducted both in fasting conditions and 1 h after glucose intake (“fed” in FIG. 13a)) and in acute dosing, i.e. only 2.5 hours from last DGA dose. From Nrf2/ARE pathway especially HO-1 and GRHPR genes were upregulated very significantly both in fasting and in fed situations after 4.5 days. Also all genes related to PGC-1a and NRF1 pathways were activated statistically very significantly in fasting after 4.5 day DGA Activation compared to 0-control. These results with relevant p-values are presented in the grey area of FIG. 13a.

Additionally, in FIG. 13a the relative basal strength of each gene expression (last column of the table) is shown. By ranking (see “RANK” in FIG. 13a) the gene expressions of the whole genome in these peripheral leukocytes, it is possible to gain knowledge on the activity of each gene in the Double Enforcement. Shown are also basal expression rates of major regulatory genes of these transcriptional pathways (Nrf2, NRF1, and NF-kB), users of NADPH (NOX4, NOXA1 and GPH) and genes related to NADPH generating (G6PD, GRHPR *), BVR*), ME1, IDH1 and PHGDH*)). Genes marked with asterisk (*) generate NADPH only when there is excess of NADH or NADP+ in the cytosol. According to the service provider a gene is active even when there are only 40 “hits” like towards Malic Enzyme (ME1).

The relative basal strength analyses were done using total RNA sequencing. It shows the basal (or 0-control) gene expression of leukocytes from S1. Genome wide HT sequencing was provided by the Technology Centre of Institute for Molecular Medicine Finland (FIMM). The RNA sequencing depth was at least 20M PE reads for each individual sample. The leukocytes were collected at the same time as the plasma samples in Example 5a.

Genes directly related to PGC-1a activation are PGC-1a and CYP2B6. The latter one is activated downstream of PGC-1a (see [11] for more information on CYP2B6). NRF1 activation related genes in FIG. 13a are GPD2 (mitochondrial glycerol-phosphate dehydrogenase), MT-CO1 (mitochondrially encoded cytochrome c oxidase I from Complex IV), and MT-CYB (mitochondrially encoded cytochrome B from Complex III) all relate directly to the mitochondrial ETS that uses O₂ and derives energy from NADH molecules and produces ATP and H2O. Downstream genes regulated by Nrf2/ARE pathways are HO-1, G6PD, GRHPR and AOX1. According to prior art [3] also NRF1 is activated simultaneously with Nrf2 especially in case of mitochondrial biogenesis.

In FIG. 13b it is further shown that HO-1, PGC-1a and CYP2B6 genes were activated also in primary human hepatocytes. This is a further remarkable proof of wide systemic non-stressful activation the UTPfsAEM and its downstream genes.

HO-1 upregulation in both the liver and in the leukocytes, shows the activation of therapeutically important HO-1 pathway both in the Local Enforcement and in the Double Enforcement.

Especially HO-1 activity and related metabolites are extremely important in acute anti-pathogen activities of the immune system. It is important to notice that in circulating peripheral leukocytes (Double Enforcement) heme degradation pathway is very relevant, because these cells handle part of the hemoglobin release from dying red blood cells in blood circulation. E.g. the basal expression of iron binding light chain ferritin is one of the largest in leukocytes; there were as many as 31166 hits for that gene (FIG. 13a). Due to its considerable volume increased intracellular HO-1 activity in peripheral leukocytes possesses significant therapeutic potential because it can effectively enhance the immune response and the functioning of the whole immune system in humans and in other vertebrates. Thus HO-1 activation is significant and relevant part of the Double Enforcement of the DGA Activation. Induced BV-BR -enzyme loop (SCHEME C) can possibly explain by new “repeatable approach” observed antiviral effects and bacterial clearance by the HO-1 activation observed already in prior art [5, 7-10]. The other important cell type for efficient therapeutic HO-1 activation is epithelial cells. Very active and thus infections prone epithelial cell linings are present in respiratory tract, in gastrointestinal tract and in the conjunctiva of the eye.

### Required Effective Dose for the Acute Infections

Use of effective dose is important in healing acute conditions like infections. From FIG. 11 one can observe that 2×200 mg oral dose mixed into water seems to be very effective in reducing fever due to infection. Notably also in thyroid and cortisol measurement (Example 5a) acute dose, i.e. last dose 1.5 hour before blood sampling, was elevated or doubled to 2×200 mg. Acute systemic inflammation and stress seems to decline efficiently with that dose.

At the same time one can observe (from FIG. 11) that 1×200 mg (3^{rd} administration for S1) is not necessarily sufficient dose. The same seems to be true for the efficacy of glaucoma therapy in FIG. 12. From FIG. 12 one can observe that sufficient and timely DGA Activation (e.g. 2×200 mg DGAcs in water), reduces intraocular pressure (IOP) but used acute dose 1*200 mg was not necessarily sufficient. Only in first measurement under the “full” DGA Activation (9.12.2014) there was very significant reduction in IOP compared to relevant control measurement (on 2^{nd} of Dec, 2014).

(Without going into details related to glaucoma, it is noted that prostaglandin F2alfa (PGF2a) analogs, e.g. latanoprost, are relatively efficient medication in reducing intra ocular pressure (IOP). They are synthesized from PGH2 in NADPH dependent reaction. Because the DGA Activation increases cellular NADPH regeneration (see SCHEME C), it is possible that the IOP reducing effect seen in FIG. 12 by the DGA Activation, is due to increase in endogenous PGF2a production from PGH2. Latanoprost was also prescribed to S1 by the ophthalmologist, but in the experiment only DGA Activation was successfully used.)

Depending on the magnitude of the inflammatory attack the above described 2×200 mg double dose should be taken 2-4 times a day for an adult patient. In some cases the needed dose could be even clearly higher (see Example if (confirmatory)). From Examples 1 b, 1 c and 1 d it can similarly be derived that in fighting against an infectious disease 2-3×200 mg for adult human (possibly several times a day) seems to be effective dose but 1×200 mg can be too little.

For animals the suitable doses per body weight can be higher, if the animal is much smaller compared to adult human being e.g. mice, broiler chicken, and possibly lower mg/kg of body weight for bigger animals like e.g. horses and cows.

### Combined Systemic Effects and Some Therapy Options for the DGA Activation

The combined results of Examples 1-5b are very convincing in showing that the DGA Activation can be efficiently used in treating infectious diseases. The anti-infectious effect is achieved by fast and non-stressful activation of cellular aerobic energy metabolism, i.e. by fast and non-stressful activation of the whole UTPfsAEM. When needed also pro-inflammatory NF-kB transcription pathway can be activated, but like presented in SCHEME A, eventually the DGA Activation leads to resolution of the infection and to decline in NF-kB activity.

In Example 5a it is shown that the capacity of adrenal and thyroid glands is liberated. This can be important in subjects experiencing some pathological condition. Liberated capacity of stimulatory hormones may be important in resolution of acute infections and fighting against inflammation in general. The body has more leeway for initiating various defense strategies. Additionally, the enhancement of metabolism and simultaneous decrease in oxidative stress protects also the liver, the kidneys and hearth as well as the respiratory tract organs and the intestinal tract, and liberates capacity of their epithelium/endothelium for their normal challenging activities. This multi-organ protection by the DGA Activation can be important in reducing unspecified infectious symptoms.

An even further positive systemic “enforcement” is the reduction in the need to shuttle substrates, e.g. lactate and amino acids, from tissues to the liver and other organs like the kidneys, and shuttle them back to the tissues. Lactate is typically converted to glucose. Nitrogen groups of amino acids are shuttled into urine or into urate in avian species and typically excreted from the body.

Similar intracellular “positive enforcement” is the reduced intracellular need to shuttle cytosolic NADH to the mitochondrial matrix for re-oxidation. Above described “additional” positive enforcements can be extremely important but at the same time they are direct or indirect consequences of successful DGA Activation either separately through the Local Enforcement or the Double Enforcement or their combined effect. Thus, it is postulated that the DGA Activation contains all these important enforcements and their existence gives further proof of concept for the DGA Activation especially in resolving acute pathological conditions. They enhance in multiple ways the function of both innate and adaptive immune systems both directly and indirectly in the fight against infections.

The D-glycerate group functions as a fast and efficient immunosuppressant “by healing the causes” in vivo in humans and in animals. Notably the mechanism of action of the DGA Activation alleviates the cause of inflammation, not just the symptoms. Competing solution e.g. synthetic glucocorticoids (hydrocortisone and other cortisol/cortisone like medicines) inhibit inflammation inter alia through inhibition of NF-kB and subsequent reduction in prostaglandin (PG) synthesis. They are used to suppress inflammation and prevent e.g. permanent damage to the tissues. Also, NSAIDs typically inhibit PG synthesis and just reduce the symptoms of inflammation like fever and pain—not the cause. Thus, the typical strategy of synthetic glucocorticoids and NSAIDs alleviates mainly or even only the symptoms of inflammation. Additionally, due to possible side effects glucocorticoids for long term use are available only as prescription drugs. Important additional advantage of the DGA Activation compared to traditional immune suppressants is that the Double Enforcement successfully activates endogenous antimicrobial activity. In this respect the use of the DGA group substances can replace antibiotics or it can enhance the action of antibiotics as a combination therapy.

Differing mechanism of action in immune suppression compared to current therapies opens tremendous possibilities for combination health products and/or combination drugs for use with the DGA Activation. On top of acting as effective and novel anti-inflammatory drug in acute, infectious and/or inflammatory diseases the DGA group substances can be effectively used in several anti-microbial therapies alone or as a combination therapy.

All in all, it can be concluded that the DGA Activation can alleviate, prevent and even heal wide range of viral, bacterial, fungal, protozoal and other infectious diseases and related inflammation. Both the Local Enforcement and the Double Enforcement are at play as can be seen from Examples 1-5b. Main intracellular pathway and molecular mechanisms underlying this remarkable healing effect are presented in SCHEME A-C.

### REFERENCES

[1] Cummings, N. W. et al. Heme oxygenase-1 regulates the immune response to influenza virus infection and vaccination in aged mice; The FASEB Journal (2012).
[2] Lindsay Hill-Batorskia, Peter Hal manna, Gabriele Neumanna and Yoshihiro Kawaokaa. The Cytoprotective Enzyme Heme Oxygenase-1 Suppresses Ebola Virus Replication; Journal of Virology (2013).
[3] Claude A. Piantadosi, Martha Sue Carraway, Abdelwahid Babiker, Hagir B. Suliman. Heme Oxygenase-1 Regulates Cardiac Mitochondrial Biogenesis via Nrf2-Mediated Transcriptional Control of Nuclear Respiratory Factor-1; Circ Res.;103:1232-1240 (2008).
[4] U.S. Pat. No. 7,666,909B2 Enhancement of alcohol metabolism.
[5] C. J. Peter Eriksson et. al. Acceleration of ethanol and acetaldehyde oxidation by d-glycerate in rats; Metabolism Volume 56, Issue 7, Pages 895-898 (2007).
[6] Hiroshi Habe, Shun Sato, Tokuma Fukuoka, Dai Kitamoto and Keiji Sakaki. Effect of Glyceric Acid Calcium Salt on the Viability of Ethanol-Dosed Gastric Cells; Journal of Oleo Science, Vol. 60, No. 11 pages 585-590 ((2011).
[7] JP2012232902A ACTIVATOR OF CELL DAMAGED BY ALCOHOL.
[8] WO2006112961A2 SYNERGISTIC SALIVATION COMPONENTS.
[9] WO2015036656A2 METHOD FOR ENHANCING ENERGY PRODUCTION AND METABOLISM IN CELLS.
[10] Hoffmann G F et al. Physiology and pathophysiology of organic acids in cerebrospinal fluid; J Inherit Metab Dis. 16(4), 648-69 (1993).
[11] Jie Gao and Wen Xie. Pregnane X Receptor and Constitutive Androstane Receptor at the Crossroads of Drug Metabolism and Energy Metabolism; Drug Metab Dispos. Dec;38(12):2091-5 (2010).
[12] Dalloul Rami A et al. Unique responses of the avian macrophage to different species of Emeria; Molecular Immunology 44 (2007) 558-566
[13] Araujo Ester C B et al. Heme oxygenase-1 activity is involved in the control of Toxoplasma gondii infection in the lung of BALB/c and C57B1/6 and in the small intestine of C57B1/6 mice; Veterinary Research 2013, 44:89
[14] Eamonn M M Quigley. Microflora Modulation of Motility; J Neurogastroenterol Motil, Vol. 17 No. 2 April, 2011
[15] V. De Pablos et al. MPTP administration increases plasma levels of acute phase proteins in non-human primates; Neuroscience Letters 463 (2009)

1-12. (canceled)

13. A method of treating, preventing or alleviating a communicable and/or an infectious disease or disorder and/or related inflammation in a subject in need thereof comprising administering an effective amount of a composition comprising one or more compounds selected from the group consisting of D-glyceric acid, DL-glyceric acid and salts and esters thereof to the subject.

14. A method of treating, preventing or alleviating a communicable and/or infectious disease or disorder and/or related inflammation, by non-stressful (=without excessive ROS generation) and simultaneous activation of cellular aerobic energy metabolism in a subject in need thereof.

15. The method of claim 13, which is for and accompanied with, respectively, activation of cellular, tissue specific and systemic immune defenses and their control against bacterial, virus, fungal or parasite (protozoa) infections and infections/inflammation arising from toxins or toxic agents, or a related disease and/or disorder.

16. The method according to claim 13, wherein the composition is designed to be administered as a replacement for antibiotics, anti-microbial agents and/or anti-inflammatory substances, or in combination therapy with antibiotics, antimicrobial agents, anti-inflammatory substances and/or other effective molecules and/or preparations.

17. The method according to claim 13, wherein the disease or disorder is an infection and/or related inflammation in epithelial and/or endothelial cells or tissues.

18. The method according to claim 13 for increasing the muscle yield per gram of nutrition and/or alternatively decreasing nutrition consumption without losing muscle mass in a subject in need thereof.

19. The method according to claim 17, wherein the epithelial cells or tissues comprise epithelium of the eyes, respiratory tract, urinary tract, reproductive tract, and/or gastrointestinal tract.

20. The method according to claim 13, wherein the disease or disorder is a viral, bacterial, protozoal, fungal and/or other infection.

21. The method according to claim 13, wherein the disease or disorder is selected from the group consisting of seasonal flu, non- seasonal flu, viral influenza, ebola, rabies, hepatitis, HIV/AIDS, herpes, polio, meningitis, conjunctivitis, keratoconjunctivitis sicca, keratitis, lacrimal gland inflammation, gastroenteritis, diarrhea, constipation, inflammatory bowel disease, including Crohn's disease and ulcerative colitis, other inflammation based disorders like diverticulosis, tuberculosis, sepsis, haemophilus influenzae (bacterial) infection, antibiotic resistant bacterial infection (e.g. MRSA), salmonella, pneumonia, tetanus, and protozoa based infections like coccidiosis, toxoplasmosis and malaria.

22. The method according to claim 13, wherein the composition is in a form of a solution, syrup, powder, ointment, mixture, capsule, tablet, or an inhalable preparation, or wherein the composition further comprises a pharmaceutically acceptable excipient.

23. The method according to claim 13, wherein the composition is in a form suitable for parenteral, oral, topical or inhalable administration.

24. The method according to claim 13, wherein the composition is part of a beverage, a food product, a functional food, a dietary supplement, or a nutritive substance.

25. The method according to claim 13, wherein the D-glyceric acid, DL-glyceric acid, or salts and esters thereof is administered at a dose of 1-2×200 mg 2-4 times a day.

26. The method according to claim 13, wherein the D-glyceric acid, DL-glyceric acid, or salts and esters thereof is administered at a dose of 5 to 10 mg/kg body weight once, twice, three or four times a day.

27. The method according to claim 13, wherein administering an effective amount of a composition comprising one or more compounds selected from the group consisting of D-glyceric acid, DL-glyceric acid and salts and esters thereof increases the muscle yield per gram of nutrition and/or alternatively decreases nutrition consumption without losing muscle mass in the subject.

28. The method according to claim 13, wherein the composition is a pharmaceutical composition.
